(19) United States
(12) Patent Application Publication
(10) Pub. No.: US 2004/0192697 A1
(43) Pub. Date: Sep. 30, 2004

(54) Pyrrolopyridine potassium channel openers

(76) Inventors: Sean C. Turner, Mannheim (DE); Tammie K. White, Gurnee, IL (US); William A. Carroll, Evanston, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/740,141

(22) Filed: Dec. 18, 2003

Related U.S. Application Data
(60) Provisional application No. 60/435,172, filed on Dec. 20, 2002.

(51) Int. Cl. C07D 471/02; A61K 31/519; A61K 31/503; A61K 31/498

(52) U.S. Cl. 514/248; 514/249; 514/265.1; 514/300; 544/236; 544/281; 544/350; 546/113

(57) ABSTRACT

Novel pyrrolopyridine compounds of formula (I),

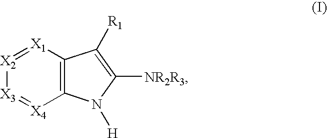

and their derivatives open potassium channels and are useful for treating a variety of diseases modulated by potassium channels.

The present application claims priority to U.S. provisional patent application No. 60/435,172, filed on Dec. 20, 2002, hereby incorporated in its entirety by reference.

### TECHNICAL FIELD

Novel pyrrolopyridine compounds and their derivatives can open potassium channels and are useful for treating a variety of diseases modulated by potassium channels.

### BACKGROUND OF INVENTION

Potassium channels play an important role in regulating cell membrane excitability. For example, when the potassium channels open, changes in the electrical potential across the cell membrane occur and result in a more polarized state. Because there exists a close relationship between potassium channels and cell excitability, many disease states associated with cell excitability can be ameliorated by regulating potassium channel receptors. Such diseases or conditions include asthma, epilepsy, male sexual dysfunction, female sexual dysfunction, pain, bladder overactivity, stroke, diseases associated with decreased skeletal blood flow such as Raynaud's phenomenon and intermittent claudication, eating disorders, functional bowel disorders, neurodegeneration, benign prostatic hyperplasia (BPH), dysmenorrhea, premature labor, alopecia, cardioprotection, coronary artery disease, angina and ischemia.

Potassium channel openers (KCOs) have been shown to act as smooth muscle relaxants, to hyperpolarize bladder cells and consequently relax bladder smooth muscle cells. Because bladder overactivity and urinary incontinence can result from the spontaneous, uncontrolled contractions of the smooth muscle of the bladder, the ability of potassium channel openers to hyperpolarize bladder cells and relax bladder smooth muscle can provide a method to ameliorate or prevent bladder overactivity, pollakiuria, bladder instability, nocturia, bladder hyperreflexia, urinary incontinence, and enuresis.

It is well known that neuronal hyperpolarization can produce analgesic effects. The opening of potassium channels by potassium channel openers and resultant hyperpolarization in the membrane of target neurons is a key mechanism in the effect of opioids. The peripheral antinociceptive effect of morphine results from activation of ATP-sensitive potassium channels, which causes hyperpolarization of peripheral terminals of primary afferents, leading to a decrease in action potential generation. Opening of K_{ATP} channels by potassium channel openers plays an important role in the antinociception mediated by alpha-2 adrenoceptors and mu opioid receptors. KCO's can also potentiate the analgesic action of both morphine and dexmedetomidine via an activation of K_{ATP} channels at the spinal cord level. Thus, potassium channel openers can hyperpolarize neuronal cells and have shown analgesic effects. Potassium channel openers therefore can be used as analgesics in the treatment of various pain states including but not limited to migraine and dyspareunia.

Calcium-activated potassium (K_{ca}) channels are a diverse group of ion channels that share a dependence on intracellular calcium ions for activity. The activity of Kₐ channels is regulated by intracellular [Ca²⁺], membrane potential and phosphorylation. On the basis of their single-channel conductances in symmetrical K⁺ solutions, K_{ca} channels are divided into three subclasses: large conductance (BK)>150 pS; intermediate conductance 50-150 pS; small conductance <50 pS. Large-conductance calcium-activated potassium (Maxi-K or BK) channels are present in many excitable cells including neurons, cardiac cells and various types of smooth muscle cells. [Singer, J. et al., Pflugers Archiv. (1987) 408, 98; Baro, I., et al., Pflugers Archiv. (1989) 414 (Suppl. 1), S 168; and Ahmed, F. et al., Br. J. Pharmacol. (1984) 83, 227]. Maxi-K (BK_{Ca}) channels are activated by an increase in intracellular calcium concentration and membrane depolarization. These channels are sensitive to blockade by iberiotoxin and charybdotoxin. The cloning of multiple splice variants of the pore-forming α-subunit (mSlo, hSlo following the nomenclature of the initially cloned Drosophila slowpoke (dSlo) calcium-activated K⁺ channel) and multiple β subunits has recently generated considerable diversity within the BK_{Ca} family. This, together with the widespread distribution of BK_{Ca} channels throughout the CNS and in peripheral tissues offers rich opportunities for discovering novel therapeutic agents as well as significant challenges in the form of tissue and organ specificity. Therapeutic applications for channel openers have focused on stroke, epilepsy, and bladder overactivity although there is evidence for utility in the treatment of asthma, hypertension, gastric hypermotility, and psychoses (Gribkoff, The Pharmacology and Molecular Biology of Large-Conductance Calcium-Activated (BK) Potassium Channels. In Advances in Pharmacology; Eds.; Academic Press: (1997); pp 319-348; Starrett, Curr. Pharm. Des. (1996), 2, 413-428).

Because of the important role played by potassium channels in Bladder overactivity and pain among many other disease states, there continues to be a need for novel compounds which open potassium channels, relax smooth muscle cells, inhibit bladder contractions and may be useful for treating diseases that can be ameliorated by opening potassium channels.

SUMMARY OF THE INVENTION

All publications, issued patents and patent applications cited herein are hereby incorporated by reference.

The present invention is directed to compounds of formula (I):

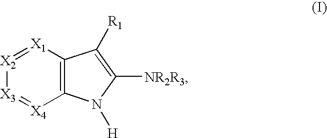

or a pharmaceutically acceptable salt, amide, ester or prodrug thereof, wherein

X₁ is selected from the group consisting of CR_{X1} and N;

X₂ is selected from the group consisting of CR_{X2} and N;

X₃ is selected from the group consisting of CR_{X3} and N;

X₄ is selected from the group consisting of CR_{X4} and N; provided that one or two of X₁, X₂, X₃, X₄ is N;

R_{X1}, R_{X2}, R_{X3} and R_{X4} are independently selected from the group consisting of hydrogen, alkenyl, alkenyloxyalkyl, alkoxyalkyl, alkoxycarbonyl, alkoxycarbonylalkyl, alkyl, alkylcarbonyl, alkylcarbonylalkyl, alkylcarbonyloxyalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cyano, cyanoalkyl, cycloalkyl, cycloalkylalkoxyalkyl, cycloalkylalkyl, cycloalkylcarbonyl, cycloalkyloxycarbonyl, cycloalkyloxyalkyl, cycloalkylalkylthioalkyl, formyl, halogen, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, hydroxy, hydroxyalkyl, nitro, R_{A}R_{B}N—, (R_{A}R_{B}N)carbonyl, (R_{A}R_{B}N)carbonylalkyl, (R_{A}R_{B}N)sulfonyl, (R_{A}R_{B}N)sulfonylalkyl, alkylsulfonylalkyl, arylalkylSalkyl, arylsulfonylalkyl, heterocyclealkylthioalkyl, HSalkyl, wherein R_{A} and R_{B} are independently selected from the group consisting of hydrogen, alkenyl, alkoxyalkyl, alkoxycarbonyl, alkoxycarbonylalkyl, alkyl, alkylcarbonyl, alkylcarbonylalkyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cycloalkyl, cycloalkylalkoxyalkyl, cycloalkylalkyl, cycloalkylcarbonyl, cycloalkyloxyalkyl, heterocycle, heterocyclealkoxyalkyl, heterocyclealkyl, heterocyclecarbonyl, heterocycleoxyalkyl, hydroxyalkyl;

R₁ is selected from the group consisting of alkylcarbonyl, carboxy, carboxyalkyl, cyano, heterocyclecarbonyl, CO₂R₄, SO₂R₄, R_{C}R_{D}N—, R_{C}R_{D}Nalkyl, alkylS-, alkylsulfonyl, (R_{C}R_{D}N)sulfonylalkyl, wherein R_{C} and R_{D} are each independently selected from the group consisting of hydrogen, alkenyl, alkoxyalkyl, alkoxycarbonyl, alkoxycarbonylalkyl, alkyl, alkylcarbonyl, alkylcarbonylalkyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cycloalkyl, cycloalkylalkoxyalkyl, cycloalkylalkyl, cycloalkylcarbonyl, cycloalkyloxyalkyl, heterocycle, heterocyclealkoxyalkyl, heterocyclealkyl, heterocyclecarbonyl, heterocycleoxyalkyl, hydroxyalkyl, and wherein R₄ is selected from the group consisting of hydrogen, alkyl and aryl;

R₂, R₃ are independently selected from the group consisting of hydrogen, alkenyl, alkenyloxyalkyl, alkoxy, alkoxyalkyl, alkoxycarbonyl, alkoxycarbonylalkyl, alkyl, alkylcarbonyl, alkylcarbonylalkyl, alkylcarbonyloxyalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cyano, cyanoalkyl, cycloalkyl, cycloalkylalkoxyalkyl, cycloalkylalkyl, cycloalkylcarbonyl, cycloalkyloxyalkyl, formyl, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, hydroxy, hydroxyalkyl, nitro, R_{E}R_{F}N—, (R_{E}R_{F}N)alkyl, (R_{E}R_{F}N)sulfonylalkyl, arylalkylSalkyl, cycloalkylalkylSalkyl, arylsulfonylalkyl, alkylSalkyl alkylsulfonylalkyl, heterocyclealkylthioalkyl, HSalkyl; wherein R_{E} and R_{F} are each independently selected from the group consisting of hydrogen, alkenyl, alkoxyalkyl, alkoxycarbonyl, alkoxycarbonylalkyl, alkyl, alkylcarbonyl, alkylcarbonylalkyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cycloalkyl, cycloalkylalkoxyalkyl, cycloalkylalkyl, cycloalkylcarbonyl, cycloalkyloxyalkyl, heterocycle, heterocyclealkoxyalkyl, heterocyclealkyl, heterocyclecarbonyl, heterocycleoxyalkyl, hydroxyalkyl; or

R₂ and R₃ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl, 1,1-dioxidothiomorpholinyl, diazepanyl, azocanyl, azonanyl, isoquinolinyl, or azabicyclononyl, wherein said heterocycle is substituted with 0, 1, or 2 substituents selected from the group consisting of hydrogen, alkenyl, alkenyloxyalkyl, alkoxy, alkoxyalkyl, alkoxycarbonyl, alkoxycarbonylalkyl, alkyl, alkylcarbonyl, alkylcarbonylalkyl, alkylcarbonyloxyalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cyano, cyanoalkyl, cycloalkyl, cycloalkylalkoxyalkyl, cycloalkylalkyl, cycloalkylcarbonyl, cycloalkyloxyalkyl, formyl, halogen, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, hydroxy, hydroxyalkyl, nitro, R_{E}R_{F}N—, (R_{E}R_{F}N)carbonyl, (R_{E}R_{F}N)carbonylalkyl, (R_{E}R_{F}N)sulfonyl, (R_{E}R_{F}N)sulfonylalkyl, alkylsulfonylalkyl, arylalkylSalkyl, arylsulfonylalkyl, heterocyclealkylthioalkyl, HSalkyl, wherein R_{E} and R_{F} are defined herein.

The present invention is also directed to the use of compounds of formula (I-V) which are potassium channel openers. Compounds which open potassium channels are useful for the treatment of disorders mediated by potassium channels. In particular one such disorder mediated by potassium channels is bladder overactivity. Accordingly, the present invention provides a method for treating bladder overactivity comprising administering a therapeutically effective amound of a compound of formula (I-V). Another disorder mediated by potassium channels is pain comprising administering a therapeutically effective amount of a compound of formula (I-V). Accordingly, the present invention provides a method for treating pain

According to another embodiment, the present invention is directed to a pharmaceutical composition comprising a therapeutically effective amount of a compound of formula (I-V) in combination with a pharmaceutically acceptable carrier

### DETAILED DESCRIPTION OF THE INVENTION

All publications, issued patents and patent applications cited herein are hereby incorporated by reference.

The present invention is directed to compounds of formula (I):

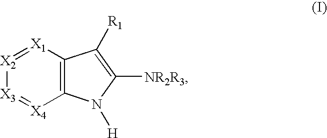

or a pharmaceutically acceptable salt, amide, ester or prodrug thereof, wherein

X₁ is selected from the group consisting of CR_{X1} and N;

X₂ is selected from the group consisting of CR_{X2} and N;

X₃ is selected from the group consisting of CR_{X3} and N;

X₄ is selected from the group consisting of CR_{X4} and N; provided that one or two of X₁, X₂, X₃, X₄ is N;

R_{X1}, R_{X2}, R_{X3} and R_{X4} are independently selected from the group consisting of hydrogen, alkenyl, alkenyloxyalkyl, alkoxyalkyl, alkoxycarbonyl, alkoxycarbonylalkyl, alkyl, alkylcarbonyl, alkylcarbonylalkyl, alkylcarbonyloxyalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cyano, cyanoalkyl, cycloalkyl, cycloalkylalkoxyalkyl, cycloalkylalkyl, cycloalkylcarbonyl, cycloalkoxycarbonyl, cycloalkyloxyalkyl, cycloalkylalkylthioalkyl, formyl, halogen, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, hydroxy, hydroxyalkyl, nitro, R_{A}R_{B}N—, (R_{A}R_{B}N)carbonyl, (R_{A}R_{B}N)carbonylalkyl, (R_{A}R_{B}N)sulfonyl, (R_{A}R_{B}N)sulfonylalkyl, alkylsulfonylalkyl, arylalkylSalkyl, arylsulfonylalkyl, heterocyclealkylthioalkyl, HSalkyl, wherein R_{A} and R_{B} are independently selected from the group consisting of hydrogen, alkenyl, alkoxyalkyl, alkoxycarbonyl, alkoxycarbonylalkyl, alkyl, alkylcarbonyl, alkylcarbonylalkyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cycloalkyl, cycloalkylalkoxyalkyl, cycloalkylalkyl, cycloalkylcarbonyl, cycloalkyloxyalkyl, heterocycle, heterocyclealkoxyalkyl, heterocyclealkyl, heterocyclecarbonyl, heterocycleoxyalkyl, hydroxyalkyl;

R₁ is selected from the group consisting of alkylcarbonyl, alkoxycarbonyl, carboxy, carboxyalkyl, cyano, heterocyclecarbonyl, CO₂R₄, SO₂R₄, R_{C}R_{D}N—, R_{C}R_{D}Nalkyl, alkylS-, alkylsulfonyl-, (R_{C}R_{D}N)sulfonylalkyl, wherein R_{C} and R_{D} are each independently selected from the group consisting of hydrogen, alkenyl, alkoxyalkyl, alkoxycarbonyl, alkoxycarbonylalkyl, alkyl, alkylcarbonyl, alkylcarbonylalkyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cycloalkyl, cycloalkylalkoxyalkyl, cycloalkylalkyl, cycloalkylcarbonyl, cycloalkyloxyalkyl, heterocycle, heterocyclealkoxyalkyl, heterocyclealkyl, heterocyclecarbonyl, heterocycleoxyalkyl, hydroxyalkyl, and wherein R₄ is selected from the group consisting of hydrogen, alkyl and aryl;

R₂, R₃ are independently selected from the group consisting of hydrogen, alkenyl, alkenyloxyalkyl, alkenyloxy(alkenyloxy)alkyl, alkoxyalkyl, alkoxycarbonyl, alkoxycarbonylalkyl, alkyl, alkylcarbonyl, alkylcarbonylalkyl, alkylcarbonyloxyalkyl, alkylsulfinylalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cyano, cyanoalkyl, cycloalkenyl, cycloalkenylalkyl, cycloalkyl, cycloalkylalkoxyalkyl, cycloalkylalkyl, cycloalkylcarbonyl, cycloalkyloxyalkyl, formyl, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, hydroxy, hydroxyalkyl, mercaptoalkyl, nitro, R_{E}R_{F}N—, (R_{E}R_{F}N)alkyl, (R_{E}R_{F}N)sulfonylalkyl, arylalkylSalkyl, cycloalkylalkylSalkyl, arylsulfonylalkyl, alkylSalkyl alkylsulfonylalkyl, heterocyclealkylthioalkyl; wherein R_{E} and R_{F} are each independently selected from the group consisting of hydrogen, alkenyl, alkoxyalkyl, alkoxycarbonyl, alkoxycarbonylalkyl, alkyl, alkylcarbonyl, alkylcarbonylalkyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cycloalkyl, cycloalkylalkoxyalkyl, cycloalkylalkyl, cycloalkylcarbonyl, cycloalkyloxyalkyl, heterocycle, heterocyclealkoxyalkyl, heterocyclealkyl, heterocyclecarbonyl, heterocycleoxyalkyl, hydroxyalkyl; or

R₂ and R₃ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl, wherein said heterocycle is substituted with 0, 1, or 2 substituents selected from the group consisting of hydrogen, alkenyl, alkenyloxyalkyl, alkoxyalkyl, alkoxycarbonyl, alkoxycarbonylalkyl, alkyl, alkylcarbonyl, alkylcarbonylalkyl, alkylcarbonyloxyalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cyano, cyanoalkyl, cycloalkyl, cycloalkylalkoxyalkyl, cycloalkylalkyl, cycloalkylcarbonyl, cycloalkyloxyalkyl, cycloalkylalkylthioalkyl, formyl, halogen, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, hydroxy, hydroxyalkyl, nitro, R_{E}R_{F}N—, (R_{E}R_{F}N)carbonyl, (R_{E}R_{F}N)carbonylalkyl, (R_{E}R_{F}N)sulfonyl, (R_{E}R_{F}N)sulfonylalkyl, alkylsulfonylalkyl, arylalkylsalkyl, arylsulfonylalkyl, heterocyclealkylthioalkyl, HSalkyl, wherein R_{E} and R_{F} are defined herein.

In one embodiment, the present invention is directed to compounds of formula (II):

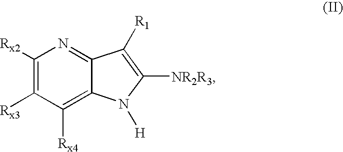

or a pharmaceutically acceptable salt, amide, ester or prodrug thereof, wherein R_{X2}, R_{X3}, R_{X4}, R₁, R₂, R₃, R_{A}, R_{B}, R_{C}, R_{D}, R_{E}, R_{F} are as defined in formula (I).

In another embodiment, the present invention is directed to compounds of formula (II), wherein R₁ is cyano and wherein R_{X2}, R_{X3}, R_{X4}, R₂, R₃, R_{A}, R_{B}, R_{C}, R_{D}, R_{E}, R_{F} are as defined in formula (I).

In another embodiment, the present invention is directed to compounds of formula (II), wherein R₁ is cyano, R₂ and R₃ are each independently selected from the group consisting of hydrogen, alkenyl, alkoxyalkyl, alkoxycarbonyl, alkoxycarbonylalkyl, alkyl, alkylcarbonyl, alkynyl, aryl, arylalkoxycarbonyl, arylalkyl, arylcarbonyl, aryloxycarbonyl, carboxyalkyl, haloalkyl, heterocycle, heterocyclealkyl, heterocyclecarbonyl, hydroxyalkyl, (R_{E}R_{F}N)alkyl and wherein R_{X2}, R_{X3}, R_{X4}, R_{A}, R_{B}, R_{C}, R_{D}, R_{E}, R_{F} are as defined in formula (I).

In another embodiment, the present invention is directed to compounds of formula (II), wherein R₁ is cyano, R₂ is alkyl, and wherein R_{X2}, R_{X3}, R_{X4}, R₃, R_{A}, R_{B}, R_{C}, R_{D}, R_{E}, R_{F} are as defined in formula (I).

In another embodiment, the present invention is directed to compounds of formula (II), wherein R₁ is cyano, R₂ is hydroxyalkyl, and wherein R_{X2}, R_{X3}, R_{X4}, R₃, R_{A}, R_{B}, R_{C}, R_{D}, R_{E}, R_{F} are as defined in formula (I).

In another embodiment, the present invention is directed to compounds of formula (II), wherein R₁ is cyano, R₂ is (R_{E}R_{F}N)alkyl, and wherein R_{X2}, R_{X3}, R_{X4}, R₃, R_{A}, R_{B}, R_{C}, R_{D}, R_{E}, R_{F} are as defined in formula (I).

In another embodiment, the present invention is directed to compounds of formula (II), wherein R₁ is cyano, R₂ and R³ together with the nitrogen that they are attached to form a heterocycle, and wherein R_{X2}, R_{X3}, R_{X4}, R_{A}, R_{B}, R_{C}, R_{D}, R_{E}, R_{F} are as defined in formula (I).

In another embodiment, the present invention is directed to compounds of formula (II), wherein R₁ is cyano, R₂ is selected from the group consisting of aryl and arylalkyl, and wherein R_{X2}, R_{X3}, R_{X4}, R₃, R_{A}, R_{B}, R_{C}, R_{D}, R_{E}, R_{F} are as defined in formula (I).

In another embodiment, the present invention is directed to compounds of formula (II), wherein R₁ is cyano, R₂ is hydrogen, and wherein R_{X2}, R_{X3}, R_{X4}, R₃, R_{A}, R_{B}, R_{C}, R_{D}, R_{E}, R_{F} are as defined in formula (I).

In another embodiment, the present invention is directed to compounds of formula (II), wherein R₁ is —CO₂R₄; R² is alkyl, and wherein R_{X2}, R_{X3}, R_{X4}, R₃, R₄, R_{A}, R_{B}, R_{C}, R_{D}, R_{E}, R_{F} are as defined in formula (I).

In another embodiment, the present invention is directed to compounds of formula (II), wherein R₁ is —CO₂R₄; R² is hydroxyalkyl, and wherein R_{X2}, R_{X3}, R_{X4}, R₃, R₄, R_{A}, R_{B}, R_{C}, R_{D}, R_{E}, R_{F} are as defined in formula (I).

In another embodiment, the present invention is directed to compounds of formula (II), wherein R₁ is —CO₂R₄; R² is (R_{E}R_{F}N)alkyl, and wherein R₂, R_{X3}, R_{X4}, R₃, R₄, R_{A}, R_{B}, R_{C}, R_{D}, R_{E}, R_{F} are as defined in formula (I).

In another embodiment, the present invention is directed to compounds of formula (II), wherein R₁ is —CO2R₄; R₂ and R³ together with the nitrogen that they are attached to form a heterocycle, and wherein R_{X2}, R_{X3}, R_{X4}, R₄, R_{A}, R_{B}, R_{C}, R_{D}, R_{E}, R_{F} are as defined in formula (I).

In another embodiment, the present invention is directed to compounds of formula (II), wherein R₁ is —CO₂R₄; R² is selected from the group consisting of aryl and arylalkyl, and wherein R_{X2}, R_{X3}, R_{X4}, R₃, R₄, R_{A}, R_{B}, R_{C}, R_{D}, R_{E}, R_{F} are as defined in formula (I).

In another embodiment, the present invention is directed to compounds of formula (II), wherein R₁ is —CO₂R₄; R² is hydrogen, and wherein R_{X2}, R_{X3}, R_{X4}, R₃, R₄, R_{A}, R_{B}, R_{C}, R_{D}, R_{E}, R_{F} are as defined in formula (I).

In one embodiment, the present invention is directed to compounds of formula (III):

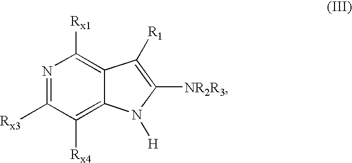

or a pharmaceutically acceptable salt, amide, ester or prodrug thereof, wherein R_{X1}, R_{X3}, R_{X4}, R₁, R₂, R₃, R_{A}, R_{B}, R_{C}, R_{D}, R_{E}, R_{F} are as defined in formula (I).

In another embodiment, the present invention is directed to compounds of formula (III), wherein R₁ is cyano and wherein R_{X1}, R_{X3}, R_{X4}, R₂, R₃, R_{A}, R_{B}, R_{C}, R_{D}, R_{E}, R_{F} are as defined in formula (I).

In another embodiment, the present invention is directed to compounds of formula (III), wherein R₁ is cyano, R₂ is selected from the group consisting of hydrogen, alkenyl, alkoxyalkyl, alkoxycarbonyl, alkoxycarbonylalkyl, alkyl, alkylcarbonyl, alkynyl, aryl, arylalkoxycarbonyl, arylalkyl, arylcarbonyl, aryloxycarbonyl, carboxyalkyl, haloalkyl, heterocycle, heterocyclealkyl, heterocyclecarbonyl, hydroxyalkyl, (R_{E}R_{F}N)alkyl, and wherein R_{X1}, R_{X3}, R_{X4}, R₃, R_{A}, R_{B}, R_{C}, R_{D}, R_{E}, R_{F} are as defined in formula (I).

In one embodiment, the present invention is directed to compounds of formula (IV):

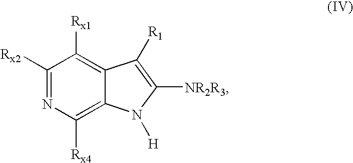

or a pharmaceutically acceptable salt, amide, ester or prodrug thereof, wherein R_{X1}, R_{X2}, R_{X4}, R₁, R₂, R₃, R_{A}, R_{B}, R_{C}, R_{D}, R_{E}, R_{F} are as defined in formula (I).

In another embodiment, the present invention is directed to compounds of formula (IV), wherein R₁ is cyano and wherein R_{X1}, R_{X2}, R_{X4}, R₂, R₃, R_{A}, R_{B}, R_{C}, R_{D}, R_{E}, R_{F} are as defined in formula (I).

In another embodiment, the present invention is directed to compounds of formula (IV), wherein R₁ is cyano, R² is selected from the group consisting of hydrogen, alkenyl, alkoxyalkyl, alkoxycarbonyl, alkoxycarbonylalkyl, alkyl, alkylcarbonyl, alkynyl, aryl, arylalkoxycarbonyl, arylalkyl, arylcarbonyl, aryloxycarbonyl, carboxyalkyl, haloalkyl, heterocycle, heterocyclealkyl, heterocyclecarbonyl, hydroxyalkyl, (R_{E}R_{F}N)alkyl, and wherein R_{X1}, R_{X2}, R_{X4}, R₃, R_{A}, R_{B}, R_{C}, R_{D}, R_{E}, R_{F} are as defined in formula (I).

In one embodiment, the present invention is directed to compounds of formula (V):

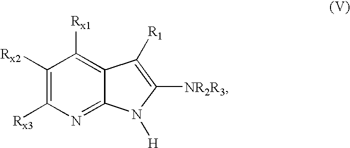

or a pharmaceutically acceptable salt, amide, ester or prodrug thereof, wherein R_{X1}, R_{X2}, R_{X3}, R₁, R₂, R₃, R_{A}, R_{B}, R_{C}, R_{D}, R_{E}, R_{F} are as defined in formula (I).

In another embodiment, the present invention is directed to compounds of formula (V), wherein R₁ is cyano and wherein R_{X1}, R_{X2}, R_{X3}, R₂, R₃, R_{A}, R_{B}, R_{C}, R_{D}, R_{E}, R_{F} are as defined in formula (I).

In another embodiment, the present invention is directed to compounds of formula (V), wherein R₁ is cyano, R² is selected from the group consisting of hydrogen, alkenyl, alkoxyalkyl, alkoxycarbonyl, alkoxycarbonylalkyl, alkyl, alkylcarbonyl, alkynyl, aryl, arylalkoxycarbonyl, arylalkyl, arylcarbonyl, aryloxycarbonyl, carboxyalkyl, haloalkyl, heterocycle, heterocyclealkyl, heterocyclecarbonyl, hydroxyalkyl, (R_{E}R_{F}N)alkyl, and wherein R_{X1}, R_{X2}, R_{X3}, R₃, R_{A}, R_{B}, R_{C}, R_{D}, R_{E}, R_{F} are as defined in formula (I).

The present invention also provides a method for the treatment or alleviation of disorders associated with BK channels, such as urinary incontinence, sexual dysfunction, male erectile dysfunction, ischemia, stroke, convulsions, epilepsy, asthma, irritable bowel syndrome, chronic pain, migraine, traumatic brain injury and spinal cord injury, which comprises administering together with a conventional adjuvant, carrier or diluent a therapeutically effective amount of a compound of formula (I) or a nontoxic pharmaceutically acceptable salt thereof. Most preferably, the compounds of Formula 1 are useful in the treatment of urinary incontinence.

### Definition of Terms

As used throughout this specification and the appended claims, the following terms have the following meanings.

The term “alkenyl,” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 1,1-dimethyl-3-butenyl, 4-pentenyl, 5-hexenyl, 2-heptenyl, 2-methyl-1-heptenyl, 3-decenyl and the like.

The term “alkenyloxy,” as used herein, refers to an alkenyl group, as defined herein, appended to the parent molecular moiety through an oxy moiety, as defined herein. Representative examples of alkenyloxy include, but are not limited to, allyloxy, 2-butenyloxy, 3-butenyloxy and the like.

The term “alkenyloxyalkyl,” as used herein, refers to a alkenyloxy group, as defined herein, appended to the parent molecular moiety through an alkyl group, as defined herein. Representative examples of alkenyloxyalkyl include, but are not limited to, (allyloxy)methyl, (2-butenyloxy)methyl and (3-butenyloxy)methyl.

The term “alkoxy,” as used herein, refers to an alkyl group, as defined herein, appended to the parent molecular moiety through an oxy moiety, as defined herein. Representative examples of alkoxy include, but are not limited to, methoxy, ethoxy, propoxy, 2-propoxy, butoxy, tert-butoxy, and the like.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, methoxymethyl, 1,1-dimethyl-3-(methoxy)propyl, and the like.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, tert-butoxycarbonyl, and the like.

The term “alkoxycarbonylalkyl,” as used herein, refers to an alkoxycarbonyl group, as defined herein, appended to the parent molecular moiety through an alkyl group, as defined herein. Representative examples of alkoxycarbonylalkyl include, but are not limited to, methoxycarbonylmethyl, ethoxycarbonylmethyl, tert-butoxycarbonylmethyl, 1,1-dimethyl-2-(methoxycarbonyl)ethyl, and the like.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 1-ethylpropyl, 2,2-dimethylpentyl, 2,3-dimethylpentyl, n-heptyl, n-octyl, n-nonyl, n-decyl, and the like.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, 1-oxopentyl, and the like.

The term “alkylcarbonylalkyl,” as used herein, refers to an alkylcarbonyl group, as defined herein, appended to the parent molecular moiety through an alkyl group, as defined herein. Representative examples of alkylcarbonylalkyl include, but are not limited to, 2-oxopropyl, 1,1-dimethyl-3-oxobutyl, 3-oxobutyl, 3-oxopentyl, and the like.

The term “alkylcarbonyloxy,” as used herein, refers to an alkylcarbonyl group, as defined herein, appended to the parent molecular moiety through an oxy moiety, as defined herein. Representative examples of alkylcarbonyloxy include, but are not limited to, acetyloxy, ethylcarbonyloxy, and the like.

The term “alkylcarbonyloxyalkyl,” as used herein, refers to an alkylcarbonyloxy group, as defined herein, appended to the parent molecular moiety through an alkyl group, as defined herein. Representative examples of alkylcarbonyloxyalkyl include, but are not limited to, acetyloxymethyl, 2-(ethylcarbonyloxy)ethyl, and the like.

The term “alkylS,” as used herein, refers to an alkyl group, as defined herein, appended to the molecular moiety through a S— group. Representative examples of alkylS- include but are not limited to methyl thio, ethyl thio and the like.

The term “alkylSalkyl,” as used herein, refers to an alkylS group, as defined herein, appended to the molecular moiety through an alkyl group. Representative examples of alkylSalkyl include but are not limited to methyl thioethyl, ethyl thioethyl and the like

The term “alkylsulfonyl-,” as used herein, refers to an alkyl group, as defined herein, appended to the parent molecular moiety through a sulfonyl-group, as defined herein. Representative examples of alkylsulfonyl include, but are not limited to, methylsulfonyl, ethylsulfonyl, and the like.

The term “alkylsulfonylalkyl,” as used herein, refers to an alkylsulfonyl group, as defined herein, appended to the parent molecular moiety through an alkyl group, as defined herein. Representative examples of alkyl sulfonylalkyl include, but are not limited to, methylsulfonylmethyl, ethylsulfonylmethyl, and the like.

The term “alkynyl,” as used herein, refers to a straight or branched chain hydrocarbon group containing from 2 to 10 carbon atoms and containing at least one carbon-carbon triple bond. Representative examples of alkynyl include, but are not limited to, acetylenyl, 1-propynyl, 2-propynyl, 3-butynyl, 2-pentynyl, 1-butynyl, and the like.

The term “aryl,” as used herein, refers to a monocyclic-ring system, or a bicyclic- or a tricyclic-fused ring system wherein one or more of the fused rings are aromatic. Representative examples of aryl include, but are not limited to, anthracenyl, azulenyl, fluorenyl, indanyl, indenyl, naphthyl, phenyl, and tetrahydronaphthyl.

The aryl groups of this invention can be substituted with 1, 2, or 3 substituents independently selected from alkenyl, alkoxy, alkoxycarbonyl, alkoxycarbonylalkyl, alkyl, alkylcarbonyl, alkylcarbonyloxy, alkylcarbonyloxyalkyl, alkyl sulfonyl, alkynyl, cyano, halo, haloalkyl, haloalkoxy, nitro, phenylalkoxycarbonyl, phenylalkoxycarbonylalkyl, phenylcarbonyloxy, phenylcarbonyloxyalkyl, phenyloxycarbonyl, phenyloxycarbonylalkyl, phenylsulfonyl, NR_{A}R_{B}, (NR_{A}R_{B})alkyl, (NR_{A}R_{B})carbonyl, (NR_{A}R_{B})carbonylalkyl, (NR_{A}RB)sulfonyl, (NR_{A}R_{B})sulfonyllalkyl, wherein R_{A} and R_{B} are each independently selected from the group consisting of hydrogen, alkyl and alkylcarbonyl.

The term “arylalkoxy,” as used herein, refers to an aryl group, as defined herein, appended to the parent molecular moiety through an alkoxy group, as defined herein. Representative examples of arylalkoxy include, but are not limited to, 2-phenylethoxy, 3-naphth-2-ylpropoxy, 5-phenylpentyloxy, and the like.

The term “arylalkoxyalkyl,” as used herein, refers to an arylalkoxy group, as defined herein, appended to the parent molecular moiety through an alkyl group, as defined herein. Representative examples of arylalkoxyalkyl include, but are not limited to, 2-phenylethoxymethyl, 2-(3-naphth-2-ylpropoxy)ethyl, 5-phenylpentyloxymethyl, and the like.

The term “arylalkoxycarbonyl,” as used herein, refers to an arylalkoxy group, as defined herein, appended to the parent molecular moiety through a carbonyl group, as defined herein. Representative examples of arylalkoxycarbonyl include, but are not limited to, benzyloxycarbonyl, naphth-2-ylmethyloxycarbonyl, and the like.

The term “arylalkoxycarbonylalkyl,” as used herein, refers to an arylalkoxycarbonyl group, as defined herein, appended to the parent molecular moiety through an alkyl group, as defined herein. Representative examples of arylalkoxycarbonylalkyl include, but are not limited to, benzyloxycarbonylmethyl, 2-(benzyloxycarbonyl)ethyl, 2-(naphth-2-ylmethyloxycarbonyl)ethyl, and the like.

The term “arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 1,1-dimethyl-2-phenylethyl, 3-phenylpropyl, 2-naphth-2-ylethyl, and the like.

The term “arylalkylS,” as used herein, refers to an arylalkyl group, as defined herein, appended to the parent molecular moiety through a thio moiety, as defined herein. Representative examples of arylalkylS include, but are not limited to, 2-phenylethylthio, 3-naphth-2-ylpropylthio, 5-phenylpentylthio, and the like.

The term “arylalkylSalkyl,” as used herein, refers to an arylalkylS group, as defined herein, appended to the parent molecular moiety through an alkyl group, as defined herein. Representative examples of arylalkylSalkyl include, but are not limited to, 2-phenylethylsulfanylmethyl, 3-naphth-2-ylpropylsulfanylmethyl, 2-(5-phenylpentylsulfanyl)ethyl, and the like.

The term “arylcarbonyl,” as used herein, refers to an aryl group, as defined herein, appended to the parent molecular moiety through a carbonyl group, as defined herein. Representative examples of arylcarbonyl include, but are not limited to, benzoyl, naphthoyl, and the like.

The term “arylcarbonylalkyl,” as used herein, refers to an arylcarbonyl group, as defined herein, appended to the parent molecular moiety through an alkyl group, as defined herein. Representative examples of arylcarbonylalkyl include, but are not limited to, 2-oxo-3-phenylpropyl, 1,1-dimethyl-3-oxo-4-phenylbutyl, and the like.

The term “arylcarbonyloxy,” as used herein, refers to an arylcarbonyl group, as defined herein, appended to the parent molecular moiety through an oxy moiety, as defined herein. Representative examples of arylcarbonyloxy include, but are not limited to, benzoyloxy, naphthoyloxy, and the like.

The term “arylcarbonyloxyalkyl,” as used herein, refers to an arylcarbonyloxy group, as defined herein, appended to the parent molecular moiety through an alkyl group, as defined herein. Representative examples of arylcarbonyloxyalkyl include, but are not limited to, benzoyloxymethyl, 2-(benzoyloxy)ethyl, 2-(naphthoyloxy)ethyl, and the like.

The term “aryloxy,” as used herein, refers to an aryl group, as defined herein, appended to the parent molecular moiety through an oxy moiety, as defined herein. Representative examples of aryloxy include, but are not limited to, phenoxy, naphthyloxy, 3-bromophenoxy, 4-chlorophenoxy, 4-methylphenoxy, 3,5-dimethoxyphenoxy, and the like.

The term “aryloxyalkyl,” as used herein, refers to an aryloxy group, as defined herein, appended to the parent molecular moiety through an alkyl group, as defined herein. Representative examples of aryloxyalkyl include, but are not limited to, 2-phenoxyethyl, 3-naphth-2-yloxypropyl, 3-bromophenoxymethyl, and the like.

The term “aryloxycarbonyl,” as used herein, refers to an aryloxy group, as defined herein, appended to the parent molecular moiety through a carbonyl moiety, as defined herein. Representative examples of aryloxycarbonyl include, but are not limited to, phenoxycarbonyl, naphthyloxycarbonyl, and the like.

The term “aryloxycarbonylalkyl,” as used herein, refers to an aryloxycarbonyl group, as defined herein, appended to the parent molecular moiety through an alkyl group, as defined herein. Representative examples of aryloxycarbonylalkyl include, but are not limited to, phenoxycarbonylmethyl, 2-(phenoxycarbonyl)ethyl, naphthyloxycarbonyl, and the like.

The term “arylsulfonyl,” as used herein, refers to an aryl group, as defined herein, appended to the parent molecular moiety through a sulfonyl group, as defined herein. Representative examples of arylsulfonyl include, but are not limited to, naphthylsulfonyl, phenylsulfonyl, 4-fluorophenylsulfonyl, and the like.

The term “arylsulfonylalkyl,” as used herein, refers to an arylsulfonyl group, as defined herein, appended to the parent molecular moiety through an alkyl group, as defined herein. Representative examples of arylsulfonylalkyl include, but are not limited to, 1,1-dimethyl-3-(phenylsulfonyl)propyl, naphthylsulfonylmethyl, 2-(phenylsulfonyl)ethyl, phenylsulfonylmethyl, 4-fluorophenylsulfonylmethyl, and the like.

The term “carbonyl,” as used herein, refers to a —C(O)— group.

The term “carbonyloxy,” as used herein, refers to a carbonyl group, as defined herein, appended to the parent molecular moiety through an oxy group, as defined herein.

The term “carbonyloxyalkyl,” as used herein, refers to a carbonyloxy group, as defined herein, appended to the parent molecular moiety through an alkyl group.

The term “carboxy,” as used herein, refers to a —CO₂H group.

The term “carboxyalkyl,” as used herein, refers to a carboxy group, as defined herein, appended to the parent molecular moiety through an alkyl group, as defined herein. Representative examples of carboxyalkyl include, but are not limited to, carboxymethyl, 2-carboxyethyl, 3-carboxypropyl, 3-carboxy-1,1-dimethylpropyl and the like.

The term “cyano,” as used herein, refers to a —CN group.

The term “cyanoalkyl,” as used herein, refers to a cyano group, as defined herein, appended to the parent molecular moiety through an alkyl group, as defined herein. Representative examples of cyanoalkyl include, but are not limited to, cyanomethyl, 2-cyanoethyl, 3-cyanopropyl, 3-cyano-1,1-dimethylpropyl, 3-cyano-1,1-diethylpropyl and the like.

The term “cycloalkyl,” as used herein, refers to a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1.^{3,7}]decane (adamantane).

The cycloalkyl groups of this invention can be substituted with 1, 2, 3, 4, or 5 substituents independently selected from alkenyl, alkoxy, alkoxycarbonyl, alkoxycarbonylalkyl, alkyl, alkylcarbonyl, alkylcarbonyloxy, alkylcarbonyloxyalkyl, alkylsulfonyl, alkynyl, cyano, halo, haloalkyl, haloalkoxy, nitro, phenylalkoxycarbonyl, phenylalkoxycarbonylalkyl, phenylcarbonyloxy, phenylcarbonyloxyalkyl, phenyloxycarbonyl, phenyloxycarbonylalkyl, phenylsulfonyl, NR_{A}R_{B}, (NR_{A}R_{B})alkyl, (NR_{A}R_{B})carbonyl, (NR_{A}R_{B})carbonylalkyl, (NR_{A}R_{B})sulfonyl, (NR_{A}R_{B})sulfonyllalkyl, wherein R_{A} and R_{B} are described herein.

The term “cycloalkylalkoxy,” as used herein, refers to a cycloalkyl group, as defined herein, appended to the parent molecular moiety through an alkoxy group, as defined herein. Representative examples of cycloalkylalkoxy include, but are not limited to, cyclopropylmethoxy, 2-cyclobutylethoxy, cyclopentylmethoxy, cyclohexylmethoxy, 4-cycloheptylbutoxy, and the like.

The term “cycloalkylalkoxyalkyl,” as used herein, refers to a cycloalkylalkoxy group, as defined herein, appended to the parent molecular moiety through an alkyl group, as defined herein. Representative examples of cycloalkylalkoxyalkyl include, but are not limited to, cyclopropylmethoxymethyl, 2-cyclobutylethoxymethyl, cyclopentylmethoxymethyl, 2-cyclohexylethoxymethyl, 2-(4-cycloheptylbutoxy)ethyl, and the like.

The term “cycloalkyloxycarbonyl,” as used herein, refers to a cycloalkyloxy group, as defined herein, appended to the parent moleculat moiety through a carbonyl group as defined herein. Representative examples of cycloalkyloxycarbonyl include, but are not limited to, cyclohexyl ester, cyclopentyl ester and the like.

The term “cycloalkylalkyl,” as used herein, refers to a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl and 4-cycloheptylbutyl, and the like.

The term “cycloalkylcarbonyl,” as used herein, refers to a cycloalkyl group, as defined herein, appended to the parent molecular moiety through a carbonyl group, as defined herein. Representative examples of cycloalkylcarbonyl include, but are not limited to, cyclopropylcarbonyl, 2-cyclobutylcarbonyl, cyclohexylcarbonyl, and the like.

The term “cycloalkyloxy,” as used herein, refers to a cycloalkyl group, as defined herein, appended to the parent molecular moiety through an oxy moiety, as defined herein. Representative examples of cycloalkyloxy include, but are not limited to, cyclohexyloxy, cyclopentyloxy, and the like.

The term “cycloalkyloxyalkyl,” as used herein, refers to a cycloalkyloxy group, as defined herein, appended to the parent molecular moiety through an alkyl group, as defined herein. Representative examples of cycloalkyloxyalkyl include, but are not limited to, 4-(cyclohexyloxy)butyl, cyclohexyloxymethyl, and the like.

The term “cycloalkylalkylS-,” as used herein, refers to a cycloalkylalkyl group, as defined herein, appended to the parent molecular moiety through a thio moiety, as defined herein. Representative examples of cycloalkylalkylS include, but are not limited to, (2-cyclohexylethyl)sulfanyl, cyclohexylmethylsulfanyl, and the like.

The term “cycloalkylalkylSalkyl,” as used herein, refers to a cycloalkylalkylthio group, as defined herein, appended to the parent molecular moiety through an alkyl group, as defined herein. Representative examples of cycloalkylalkylSalkyl include, but are not limited to, 2-[(2-cyclohexylethyl)sulfanyl]ethyl, (2-cyclohexylethyl)sulfanylmethyl, and the like.

The term “formyl,” as used herein, refers to a —C(O)H group.

The term “halo” or “halogen,” as used herein, refers to —Cl, —Br, —I or —F.

The term “haloalkenyl,” as used herein, refers to at least one halogen, as defined herein, appended to the parent molecular moiety through an alkenyl group, as defined herein. Representative examples of haloalkenyl include, but are not limited to, 2,2-dichloroethenyl, 2,2-difluoroethenyl, 5-chloropenten-2-yl, and the like.

The term “haloalkyl,” as used herein, refers to at least one halogen, as defined herein, appended to the parent molecular moiety through an alkyl group, as defined herein. Representative examples of haloalkyl include, but are not limited to, chloromethyl, trichloromethyl, 1,1-dichloroethyl, 2-fluoroethyl, trifluoromethyl, 2,2,2-trifluoroethyl, 2,2,2-trifluoro-1-(trifluoromethyl)-1-(methyl)ethyl, pentafluoroethyl, 2-chloro-3-fluoropentyl, and the like.

The term “haloalkynyl,” as used herein, refers to at least one halogen, as defined herein, appended to the parent molecular moiety through an alkyne group, as defined herein. Representative examples of haloalkynyl include, but are not limited to, 1-chlorobut-2-ynyl, 1,1-dichloropent-2-ynyl, 7,7-dichloro-5-methylhept-3-ynyl, and the like.

The term “haloalkylcarbonyl,” as used herein, refers to a haloalkyl group, as defined herein, appended to the parent molecular moiety through a carbonyl group, as defined herein. Representative examples of haloalkylcarbonyl include, but are not limited to, chloromethylcarbonyl, trichloromethylcarbonyl, trifluoromethylcarbonyl, and the like.

The term “haloalkylsulfonyl,” as used herein, refers to a haloalkyl group, as defined herein, appended to the parent molecular moiety through a sulfonyl group, as defined herein. Representative examples of haloalkylsulfonyl include, but are not limited to, chloromethylsulfonyl, trichloromethylsulfonyl, trifluoromethylsulfonyl, and the like.

The term “heterocycle,” as used herein, refers to a monocyclic or a bicyclic ring system. Monocyclic ring systems are exemplified by any 5 or 6 membered ring containing 1, 2, 3, or 4 heteroatoms independently selected from oxygen, nitrogen and sulfur. The 5-membered ring has from 0-2 double bonds and the 6-membered ring has from 0-3 double bonds. Representative examples of monocyclic ring systems include, but are not limited to, azetidine, azepine, aziridine, diazepine, 1,3-dioxolane, dioxane, 1 ,3-dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzotriazole, benzodioxine, 1,3-benzodioxole, cinnoline, indazole, indole, indoline, indolizine, naphthyridine, isobenzofuran, isobenzothiophene, isoindole, isoindoline, 1-isoindolinone, isoquinoline, 1-isoquinolinone, phthalazine, pyranopyridine, quinoline, quinolizine, quinoxaline, quinazoline, tetrahydroisoquinoline, tetrahydroquinoline, and thiopyranopyridine.

The heterocycle groups of this invention can be substituted with 1, 2, or 3 substituents independently selected from alkenyl, alkoxy, alkoxycarbonyl, alkoxycarbonylalkyl, alkyl, alkylcarbonyl, alkylcarbonyloxy, alkylcarbonyloxyalkyl, alkylsulfonyl, alkynyl, cyano, halo, haloalkyl, haloalkoxy, nitro, phenylalkoxycarbonyl, phenylalkoxycarbonylalkyl, phenylcarbonyloxy, phenylcarbonyloxyalkyl, phenyloxycarbonyl, phenyloxycarbonylalkyl, phenylsulfonyl, R_{A}R_{B}N—, (R_{A}R_{B}N)alkyl, (R_{A}R_{B}N)carbonyl, (R_{A}R_{B}N)carbonylalkyl, (R_{A}R_{B}N)sulfonyl, (R_{A}R_{B}N)sulfonylalkyl, wherein R_{A} and R_{B} described herein.

The term “heterocyclealkoxy,” as used herein, refers to a heterocycle group, as defined herein, appended to the parent molecular moiety through an alkoxy group, as defined herein. Representative examples of heterocyclealkoxy include, but are not limited to, 2-pyrid-3-ylethoxy, 3-quinolin-3-ylpropoxy, 5-pyrid-4-ylpentyloxy, and the like.

The term “heterocyclealkoxyalkyl,” as used herein, refers to a heterocyclealkoxy group, as defined herein, appended to the parent molecular moiety through an alkyl group, as defined herein. Representative examples of heterocyclealkoxyalkyl include, but are not limited to, 2-pyrid-3-ylethoxymethyl, 2-(3-quinolin-3-ylpropoxy)ethyl, 5-pyrid-4-ylpentyloxymethyl, and the like.

The term “heterocyclealkyl,” as used herein, refers to a heterocycle, as defined herein, appended to the parent molecular moiety through an alkyl group, as defined herein. Representative examples of heterocyclealkyl include, but are not limited to, pyrid-3-ylmethyl, pyrimidin-5-ylmethyl, and the like.

The term “heterocyclealkylthio,” as used herein, refers to a heterocyclealkyl group, as defined herein, appended to the parent molecular moiety through a thio moiety, as defined herein. Representative examples of heterocyclealkylthio include, but are not limited to, 2-pyrid-3-ylethysulfanyl, 3-quinolin-3-ylpropysulfanyl, 5-pyrid-4-ylpentylsulfanyl, and the like.

The term “heterocyclealkylthioalkyl,” as used herein, refers to a heterocyclealkylthio group, as defined herein, appended to the parent molecular moiety through an alkyl group, as defined herein. Representative examples of heterocyclealkylthioalkyl include, but are not limited to, 2-pyrid-3-ylethysulfanylmethyl, 2-(3-quinolin-3-ylpropysulfanyl)ethyl, 5-pyrid-4-ylpentylsulfanylmethyl, and the like.

The term “heterocyclecarbonyl,” as used herein, refers to a heterocycle, as defined herein, appended to the parent molecular moiety through a carbonyl group, as defined herein. Representative examples of heterocyclecarbonyl include, but are not limited to, pyrid-3-ylcarbonyl, quinolin-3-ylcarbonyl, thiophen-2-ylcarbonyl, and the like.

The term “heterocycleoxy,” as used herein, refers to a heterocycle group, as defined herein, appended to the parent molecular moiety through an oxy moiety, as defined herein. Representative examples of heterocycleoxy include, but are not limited to, pyrid-3-yloxy, quinolin-3-yloxy, and the like.

The term “heterocycleoxyalkyl,” as used herein, refers to a heterocycleoxy group, as defined herein, appended to the parent molecular moiety through an alkyl group, as defined herein. Representative examples of heterocycleoxyalkyl include, but are not limited to, pyrid-3-yloxymethyl, 2-quinolin-3-yloxyethyl, and the like.

The term “heterocyclesulfonyl,” as used herein, refers to a heterocycle group, as defined herein, appended to the parent molecular moiety through a sulfonyl group, as defined herein. Representative examples of hetercyclesulfonyl include, but are not limited to pyrid-3-ylsulfonyl, quinolin-3-ylsulfonyl, thiophen-2-ylsulfonyl, and the like.

The term “HSalkyl,” as used herein, refers to an HS group appended to the parent molecular moiety through an alkyl group, as defined herein.

The term “hydroxy,” as used herein, refers to an —OH group.

The term “hydroxyalkyl,” as used herein, refers to at least one hydroxy groups, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypropyl, 2-ethyl-4-hydroxyheptyl, 2-hydroxy-1,1-dimethylethyl, 3-hydroxy-1,1-dimethylpropyl, and the like.

The term “mercapto,” as used herein, refers to a —SH group.

The term “mercaptoalkyl,” as used herein, refers to a mercapto group, as defined herein, appended to the parent molecular moiety through an alkyl group, as defined herein. Representative examples of mercaptoalkyl include, but are not limited to, 2-sulfanylethyl, 3-sulfanylpropyl, and the like.

The term “R_{A}R_{B}N”, as used herein, refers to R_{A} and R_{B}, as defined herein, which are appended to the parent molecular moiety through a nitrogen atom. Representative examples of R_{A}R_{B}N include, but are not limited to, methylamine, dimethylamine, phenylamine, and the like.

The term “(R_{A}R_{B}N)carbonyl,” as used herein, refers to a R_{A}R_{B}N group as defined herein, attached to the parent molecular moiety through a carbonyl group as defined herein. Representative examples of (R_{A}R_{B}N)carbonyl include, but are not limited to, methylformido, dimethylformido, phenylformido, benzylformido, and the like.

The term “(R_{A}R_{B}N)carbonylalkyl”, as used herein, refers to a (R_{A}R_{B}N)carbonyl group as defined herein, appended to the parent molecular moiety through an alkyl group as defined herein. Representative examples of (R_{A}R_{B}N)carbonylalkyl include, but are not limited to, N-methylacetamido, N-dimethylacetamido, N-phenylacetamido, benzylacetamido, and the like.

The term, “(R_{A}R_{B}N)sulfonyl,” as used herein, refers to a (R_{A}R_{B}N) group, as defined herein, appended to the parent molecular moiet through an sulfonyl group, as defined herein. Representative examples of (R_{A}R_{B}N)sulfonyl include, but are not limited to, methylsulfonyl, dimethylsulfonyl, phenylsulfonyl, benzylsulfonyl, and the like.

The term, “(R_{A}R_{B}N)sulfonylalkyl,” as used herein, refers to a (R_{A}R_{B}N)sulfonyl group, as defined herein, appended to the parent molecular moiet through an alkyl group, as defined herein.

The term “R_{C}R_{D}N”, as used herein, refers to R_{C} and R_{D}, as defined herein, which are appended to the parent molecular moiety through a nitrogen atom. Representative examples of R_{A}R_{B}N include, but are not limited to, methylamine, dimethylamine, phenylamine, and the like.

The term “R_{C}R_{D}Nalkyl,” as used herein, refers to a R_{C}R_{D}N, as defined herein appended to the parent molecular moiety through an alkyl group, as defined herein.

The term, “(R_{C}R_{D}N)sulfonyl,” as used herein, refers to a (R_{C}R_{D}N) group, as defined herein, appended to the parent molecular moiet through an sulfonyl group, as defined herein. Representative examples of (R_{C}R_{D}N)sulfonyl include, but are not limited to, methylsulfonyl, dimethylsulfonyl, phenylsulfonyl, benzylsulfonyl, and the like.

The term, “(R_{C}R_{D}N)sulfonylalkyl,” as used herein, refers to a (R_{C}R_{D}N)sulfonyl group, as defined herein, appended to the parent molecular moiet through an alkyl group, as defined herein.

The term “R_{E}R_{F}N”, as used herein, refers to R_{E} and R_{F}, as defined herein, which are appended to the parent molecular moiety through a nitrogen atom. Representative examples of R_{A}R_{B}N include, but are not limited to, methylamine, dimethylamine, phenylamine, and the like.

The term “R_{E}R_{F}Nalkyl,” as used herein, refers to a R_{E}R_{F}N, as defined herein appended to the parent molecular moiety through an alkyl group, as defined herein.

The term, “(R_{E}R_{F}N)sulfonyl,” as used herein, refers to a (R_{E}R_{F}N) group, as defined herein, appended to the parent molecular moiet through an sulfonyl group, as defined herein. Representative examples of (R_{E}R_{F}N)sulfonyl include, but are not limited to, methylsulfonyl, dimethylsulfonyl, phenylsulfonyl, benzylsulfonyl, and the like.

The term, “(R_{E}R_{F}N)sulfonylalkyl,” as used herein, refers to a (R_{E}R_{F}N)sulfonyl group, as defined herein, appended to the parent molecular moiet through an alkyl group, as defined herein.

The term “(R_{E}R_{F}N)carbonyl”, as used herein, refers to a (R_{E}R_{F}N) group as defined herein, appended to the parent molecular moiety through a carbonyl group as defined herein. Representative examples of (R_{E}R_{F}N)carbonyl include, but are not limited to, methylformido, dimethylformido, phenylformido, benzylformido, and the like.

The term “(R_{E}R_{F}N)carbonylalkyl”, as used herein, refers to a (R_{E}R_{F}N)carbonyl group as defined herein, appended to the parent molecular moiety through an alkyl group as defined herein. Representative examples of (R_{E}R_{F}N)carbonylalkyl include, but are not limited to, N-methylacetamido, N-dimethylacetamido, N-phenylacetamido, benzylacetamido, and the like.

The term “nitro,” as used herein, refers to a —NO₂ group.

The term “oxo,” as used herein, refers to a (═O) moiety.

The term “oxy,” as used herein, refers to a (—O—) moiety.

The term “oxycarbonyl,” as used herein, refers to an oxy group as defined herein, appended to the parent molecular moiety through a carbonyl group as defined herein.

The term “oxycarbonylalkyl,” as used herein, refers to an oxycarbonyl group as defined herein, appended to the parent molecular moiety through an alkyl group as defined herein.

The term “phenylalkoxycarbonyl,” as used herein, refers to a phenyl group as defined herein, appended to the parent molecular moiety through an alkoxycarbonyl group as defined herein. Representative examples of phenylalkoxycarbonyl include, but are not limited to benzyl formyl, 1-naphthylmethyl formyl, 3-phenylpropyl formyl, and the like.

The term “phenylalkoxycarbonylalkyl,” as used herein, refers to a phenyl group as defined herein, appended to the parent molecular moiety through an alkoxycarbonylalkyl group as defined herein. Representative examples of phenylalkoxycarbonylalkyl include, but are not limited to benzyl propionyl, naphthyl propionyl, 1-phenylethyl propionyl, and the like.

The term “phenylcarbonyloxy,” as used herein, refers to a phenyl group as defined herein, appended to the parent molecular moiety through a carbonyloxy group as defined herein. Representative examples of phenylcarbonyloxy include, but are not limited to benzoyl, 1-naphthoyl, and the like.

The term “phenylcarbonyloxyalkyl,” as used herein, refers to a phenyl group as defined herein, appended to the parent molecular moiety through a carbonyloxyalkyl group as defined herein. Representative examples of phenylcarbonyloxyalkyl include, but are not limited to ethyl benzoyl, propyl benzoate and the like.

The term “phenyloxycarbonyl,” as used herein, refers to a phenyl group as defined herein, appended to the parent molecular moiety through a oxycarbonyl group as defined herein. Representative examples of phenyloxycarbonyl include, but are not limited to phenyl formyl, naphthyl formyl, and the like.

The term “phenyloxycarbonylalkyl,” as used herein, refers to a phenyl group as defined herein, appended to the parent molecular moiety through an oxycarbonylalkyl group as defined herein. Representative examples of phenyloxycarbonylalkyl include, but are not limited to phenyl propionate, naphthyl propionyl, and the like.

The term “phenylsulfonyl,” as used herein, refers to a phenyl group as defined herein, appended to the parent molecular moiety through a sulfonyl group as defined herein. Representative examples of phenylsulfonyl include, but are not limited to phenyl sulfonyl, naphthyl sulfonyl, and the like.

The term “sulfonyl,” as used herein, refers to a —SO₂— group.

The term “thio,” as used herein, refers to a —S— group.

The term “tautomer,” as used herein, refers to a proton shift from one atom of a molecule to another atom of the same molecule. The present invention contemplates that particular compounds may exist as tautomers and are contemplated within the scope of the present invention.

Compounds of the present invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30. The present invention contemplates various stereoisomers and mixtures thereof and are specifically included within the scope of this invention. Stereoisomers include enantiomers and diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns. Specific compounds of formula (I) include, but are not limited to:

2-Dimethylamino-1H-pyrrolo[3,2-b]pyridine-3-carbonitrile;

2-Diethylamino-1H-pyrrolo[3,2-b]pyridine-3-carbonitrile;

2-[(2-Dimethylamino-ethyl)-methyl-amino]-1H-pyrrolo[3,2-b]pyridine-3-carbonitrile;

2-[(2-Hydroxy-ethyl)-propyl-amino]-1H-pyrrolo [3,2-b]pyridine-3-carbonitrile;

2-Pyrrolidin-1-yl-1H-pyrrolo[3,2-b]pyridine-3-carbonitrile;

2-(2,5-Dihydro-pyrrol-1-yl)-1H-pyrrolo[3,2-b]pyridine-3-carbonitrile;

(R)-2-(3-Hydroxy-pyrrolidin-1-yl)-1H-pyrrolo[3,2-b]pyridine-3-carbonitrile;

(S)-2-(3-Hydroxy-pyrrolidin-1-yl)-1H-pyrrolo[3,2-b]pyridine-3-carbonitrile;

(R)-2-(2-Hydroxymethyl-pyrrolidin-1-yl)-1H-pyrrolo[3,2-b]pyridine-3-carbonitrile;

(S)-2-(2-Hydroxymethyl-pyrrolidin-1-yl)-1H-pyrrolo[3,2-b]pyridine-3-carbonitrile;

(S)-1-(3-Cyano-1H-pyrrolo[3,2-b]pyridin-2-yl)-pyrrolidine-2-carboxylic acid methyl ester;

(R)-1-(3-Cyano-1H-pyrrolo[3,2-b]pyridin-2-yl)-pyrrolidine-2-carboxylic acid methyl ester;

(R)-[1-(3-Cyano-1H-pyrrolo[3,2-b]pyridin-2-yl)-pyrrolidin-3-yl]-carbamic acid tert-butyl ester;

(S)-[1-(3-Cyano-1H-pyrrolo[3,2-b]pyridin-2-yl)-pyrrolidin-3-yl]-carbamic acid tert-butyl ester;

(R,R)-2-(2,5-Dimethyl-pyrrolidin-1-yl)-1H-pyrrolo[3,2-b]pyridine-3-carbonitrile;

(S, S)-2-(2,5-Bis-methoxymethyl-pyrrolidin-1-yl)-1H-pyrrolo[3,2-b]pyridine-3-carbonitrile;

2-Imidazol-1-yl-1H-pyrrolo[3,2-b]pyridine-3-carbonitrile;

2-Piperidin-1-yl-1H-pyrrolo[3,2-b]pyridine-3-carbonitrile;

2-(3-Hydroxy-piperidin-1-yl)-1H-pyrrolo[3,2-b]pyridine-3-carbonitrile;

2-(4-Hydroxy-piperidin-1-yl)-1H-pyrrolo[3,2-b]pyridine-3-carbonitrile;

1-(3-Cyano-1H-pyrrolo[3,2-b]pyridin-2-yl)-piperidine-4-carboxylic acid methyl ester;

2-(4-Methyl-piperazin-1-yl)-1H-pyrrolo[3,2-b]pyridine-3-carbonitrile;

2-Morpholin-4-yl-1H-pyrrolo[3,2-b]pyridine-3-carbonitrile;

2-Azepan-1-yl-1H-pyrrolo[3,2-b]pyridine-3-carbonitrile;

2-(4-Methyl-[1,4]diazepan-1-yl)-1H-pyrrolo[3,2-b]pyridine-3-carbonitrile;

2-Azocan-1-yl-1H-pyrrolo[3,2-b]pyridine-3-carbonitrile;

2-Azonan-1-yl-1H-pyrrolo[3,2-b]pyridine-3-carbonitrile;

2-(Octahydro-isoquinolin-2-yl)-1H-pyrrolo[3,2-b]pyridine-3-carbonitrile;

2-(3-Aza-bicyclo[3.2.2]non-3-yl)-1H-pyrrolo[3,2-b]pyridine-3-carbonitrile;

2-Phenylamino-1H-pyrrolo[3,2-b]pyridine-3-carbonitrile;

2-Benzylamino-1H-pyrrolo[3,2-b]pyridine-3-carbonitrile; and

2-Amino-1H-pyrrolo[3,2-b]pyridine-3-carboxylic acid ethyl ester.

### Preparation of Compounds of the Invention

The compounds and processes of the present invention will be better understood in connection with the following synthetic schemes and methods which illustrate a means by which the compounds of the invention can be prepared.

The compounds of this invention may be prepared by a variety of synthetic routes. Representative procedures are shown in Schemes 1-9.

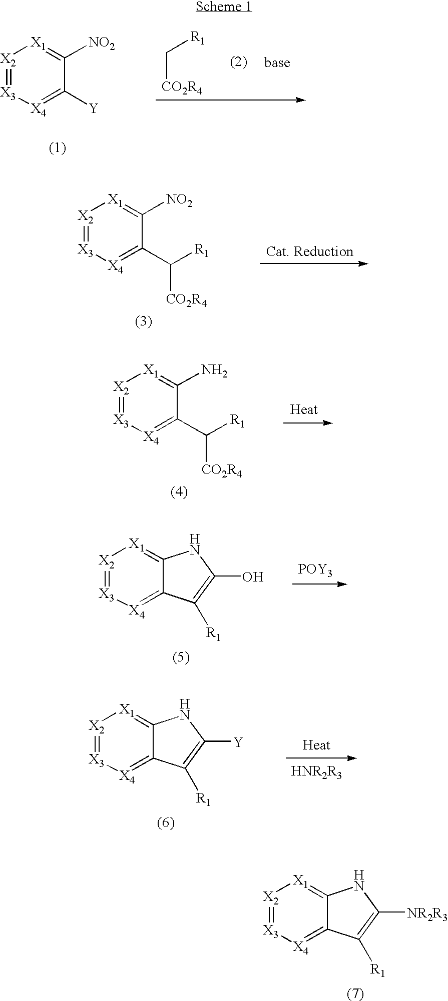

As shown in Scheme 1, pyrrolopyridines of general formula (7), wherein R₁, R₂, R₃, R₄, X₁, X₂, X₃, X₄ are as defined in formula (I), and Y is defined as bromine or chlorine, may be prepared using the strategy outlined above. Nitrohalopyridines of general formula (1) may be treated with enolates of α-substituted esters of general formula (2), formed by treatment with a suitable base such as sodium hydride or potassium t-butoxide, to provide pyridyl esters of general formula (3). Pyridyl esters of general formula (3) can be reduced to the corresponding aminopyridyl derivatives of general formula (4) by catalytic hydrogenation employing a catalyst such as palladium on carbon. Aminopyridyl derivatives of general formula (4) may by cyclized, by heating in a high boiling point solvent such as xylene, to give pyrrolopyridines of general formula (5). Pyrrolopyridines of general formula (5) may be treated with chlorinating or brominating agents (for example phosphorus oxychloride) either neat or in the presence of an appropriate solvent, to give halopyrrolopyridines of general formula (6). Halopyrrolopyridines of general formula (6) can be treated with primary or secondary amines to give aminopyrrolopyridines of general formula (7).

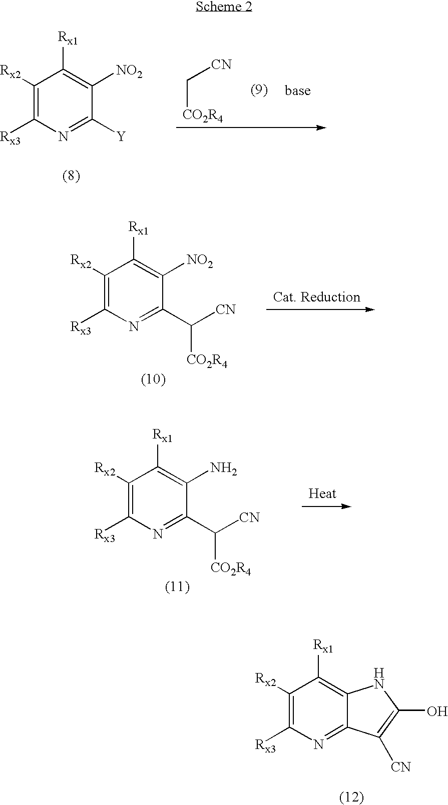

As shown in Scheme 2, pyrrolopyridines of general formula (12), wherein R₁, R₄, Rₓ₁, Rₓ₂, Rₓ₃ are as defined in formula (I), may be prepared using the strategy outlined above. 3-Nitropyridines of general formula (8) may be treated with enolates of α-cyanoesters of general formula (9), formed by treatment with a suitable base such as sodium hydride or potassium t-butoxide, in a solvent such as t-butanol [Finch, N., Robison, M. M. and Valerio, M. P., J. Org. Chem., 1972, 37 (1), 51-54] to provide pyridyl α-cyanoesters of general formula (10). Pyridyl α-cyanoesters of general formula (10) can be reduced to the corresponding aminopyridyl derivatives of general formula (11) by catalytic hydrogenation employing a catalyst such as palladium on carbon [R. W. Daisley, J. R. Hanbali, J. Heterocyclic Chem., 1983, 20, 999]. Aminopyridyl derivatives of general formula (11) may by cyclized, by heating in a high boiling point solvent such as xylene, to give pyrrolopyridines of general formula (12).

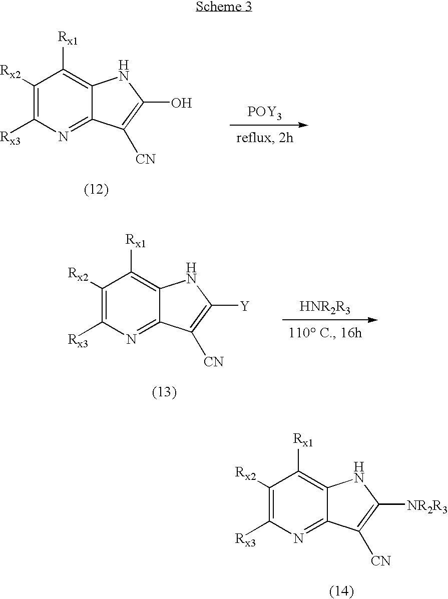

As shown in Scheme 3, aminopyrrolopyridines of general formula (14), wherein R₁, R₂, R₃, Rₓ₁, Rₓ₂, and Rₓ₃ are as defined in formula (I), may be prepared using the strategy outlined above. Pyrrolopyridines of general formula (12) may be treated with chlorinating or brominating agents (for example phosphorus oxychloride) either neat or in the presence of an appropriate solvent, to give halopyrrolopyridines of general formula (13). Halopyrrolopyridines of general formula (13) can be treated with primary or secondary amines to give aminopyrrolopyridines of general formula (14).

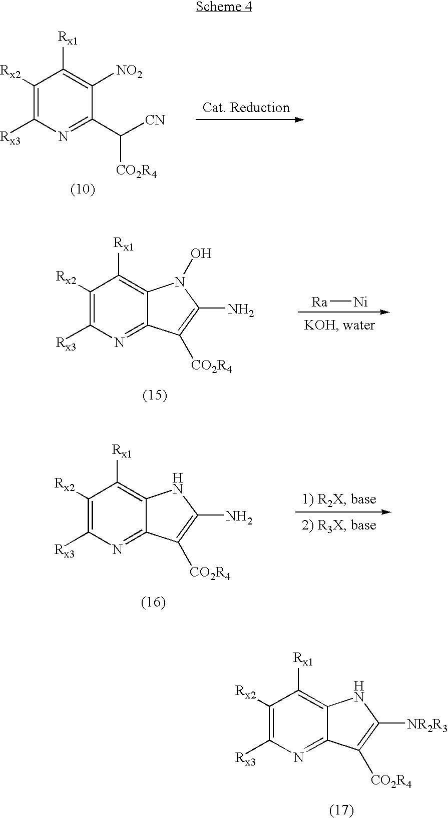

As shown in Scheme 4, aminopyrrolopyridines of general formula (17), wherein R₁, R₂, R₃, R₄, Rₓ₁, Rₓ₂, and Rₓ₃ are as defined in formula (I), may be prepared using the strategy outlined above. Pyridyl α-cyanoesters of general formula (10) can be reduced to the corresponding N-hydroxy aminopyrrolopyridines derivatives of general formula (15) by catalytic hydrogenation employing a catalyst such as palladium on carbon. N-hydroxy aminopyrrolopyridines derivatives of general formula (15) may undergo N—O cleavage by a hydrogen source such as be Raney nickel or a one-electron a reducing agent such as samarium iodide to give aminopyrrolopyridines of general formula (16). Finally, aminopyrrolopyridines of general formula (16) can be N-alkylated with aryl or alkyl halides to give aminopyrrolopyridines of general formula (17).

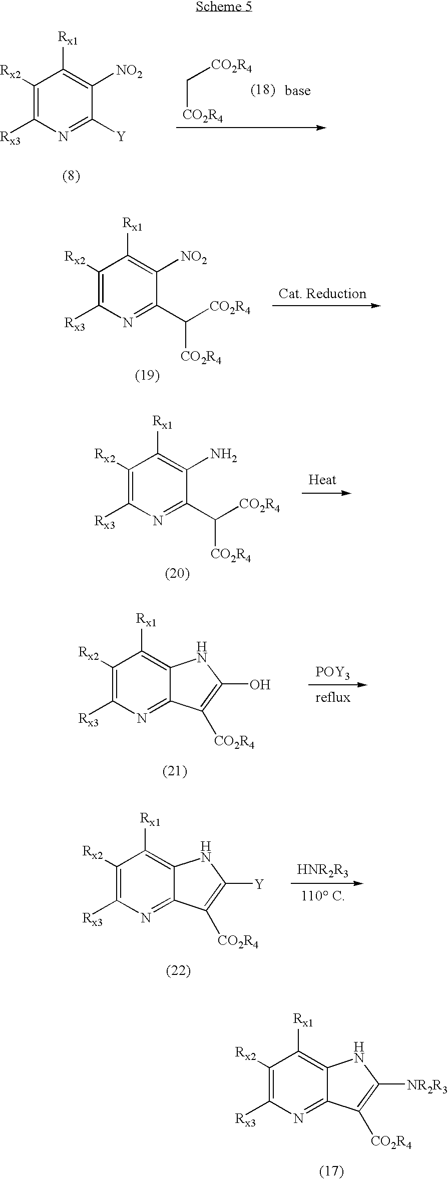

As shown in Scheme 5, aminopyrrolopyridines of general formula (17), wherein R₁, R₂, R₃, R₄, Rₓ₁, Rₓ₂, Rₓ₃ are as defined in formula (I), may be prepared using the strategy outlined above. 3-Nitropyridines of general formula (8) may be treated with enolates of malonate diesters of general formula (18), formed by treatment with a suitable base such as sodium hydride or potassium t-butoxide, in a solvent such as t-butanol [B. A. J. Clark, M. S. El-Bakoush, J. Patrick, J. Chem. Soc., Perkin Trans. I, 1974, 1531] to provide pyridyl diesters of general formula (19). Pyridyl diesters of general formula (19) can be reduced to the corresponding aminopyridyl derivatives of general formula (20) by catalytic hydrogenation employing a catalyst such as palladium on carbon. Aminopyridyl derivatives of general formula (20) may by cyclized, by heating in a high boiling point solvent such as xylene, to give pyrrolopyridines of general formula (21). Pyrrolopyridines of general formula (21) may be treated with chlorinating or brominating agents (for example phosphorus oxychloride) either neat or in the presence of an appropriate solvent, to give halopyrrolopyridines of general formula (22). Halopyrrolopyridines of general formula (22) can be treated with primary or secondary amines to give aminopyrrolopyridines of general formula (17).

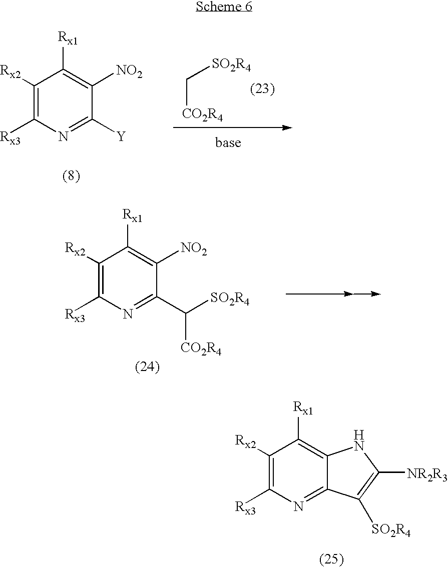

As shown in Scheme 6, pyrrolopyridines of general formula (25), wherein R₁, R₂, R₃, R₄, Rₓ₁, Rₓ₂, Rₓ₃ are as defined in formula (I), may be prepared using the strategy outlined above. 3-Nitropyridines of general formula (8) may be treated with enolates of α-sulfonylesters of general formula (23), formed by treatment with a suitable base such as sodium hydride or potassium t-butoxide in a solvent such as DMF [R. Goumont, N. Faucher, G. Moutiers, T. Gilles, M. Tordeux, C. Wakselman, Synthesis, 1997, 6, 691] to provide pyridyl α-sulfonylesters of general formula (24). Pyridyl α-sulfonylesters of general formula (24) may be converted by application of the processes described in Schemes 1 and 2 to give pyrrolopyridines of general formula (25).

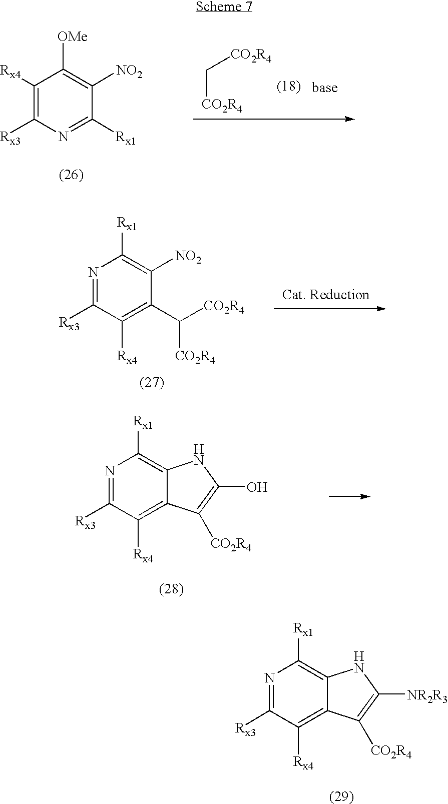

As shown in Scheme 7, pyrrolopyridines of general formula (29), wherein R₁, R₂, R₃, R₄, Rₓ₁, Rₓ₃, and Rₓ₄ are as defined in formula (I), may be prepared using the strategy outlined above. 3-Nitropyridines of general formula (26) may be treated with enolates of malonate diesters of general formula (18), formed by treatment with a suitable base such as sodium hydride or potassium t-butoxide, in a solvent such as t-butanol [O. Bremer, Ann. Chem., 1937, 529, 290] to provide pyridyl diesters of general formula (27). Pyridyl diesters of general formula of general formula (27) can be reduced to the corresponding pyrrolopyridines of general formula (28) by catalytic hydrogenation employing a catalyst such as palladium on carbon [R. W. Daisley, J. R. Hanbali, J. Heterocyclic Chem., 1983, 20, 999]. Pyrrolopyridines of general formula (28) may be converted to aminopyrrolopyridines of general formula (29) by the sequence described in Scheme 1.

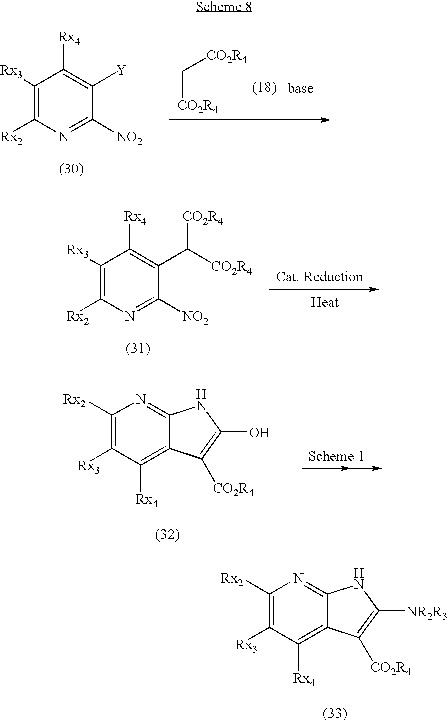

As shown in Scheme 8, pyrrolopyridines of general formula (33), wherein R₁, R₂, R₃, R₄, Rₓ₂, Rₓ₃, Rₓ₄ are as defined in formula (I), may be prepared using the strategy outlined above. 2-Nitropyridines of general formula (30) may be treated with enolates of malonate diesters of general formula (18), formed by treatment with a suitable base such as sodium hydride or potassium t-butoxide, in a solvent such as t-butanol to provide pyridyl diesters of general formula (31). Pyridyl diesters of general formula of general formula (31) can be reduced to the corresponding pyrrolopyridines of general formula (32) by catalytic hydrogenation employing a catalyst such as palladium on carbon. Pyrrolopyridines of general formula (32) may be converted to aminopyrrolopyridines of general formula (33) by the sequence described in Scheme 1.

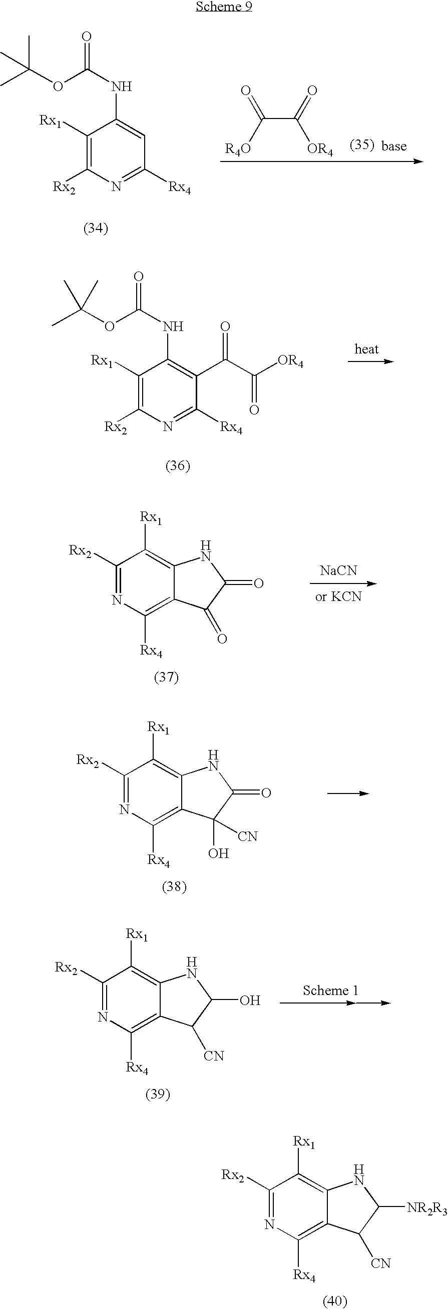

As shown in Scheme 9, pyrrolopyridines of general formula (40), wherein R₁, R₂, R₃, R₄, Rₓ₁, Rₓ₂, Rₓ₄ are as defined in formula (I), may be prepared using the strategy outlined above. N-BOC 4-aminopyridines of general formula (34) may be ortho-lithiated by treatment with an appropriate base (such as n-BuLi) and the resulting anion reacted with an oxalic acid derivative of general formula (35) to provide pyridyl a-ketoesters of general formula (36). Pyridyl α-ketoesters of general formula (36) can cyclized to the corresponding azaisatins of general formula (37) by acid cleavage of the BOC group followed by thermal cyclization. Azaisatins of general formula (37) may be converted to the 3-cyano-3-hydroxy derivatives (38) by addition of a metal cyanide. 3-Cyano-3-hydroxy derivatives (38) may be converted to the 3-cyano derivatives (39) by derivatization of the 3-hydroxy (e.g. as an acetoxy group) followed by catalytic hydrogenation to give the 3-cyano derivatives (39). This can be converted to aminopyrrolopyridines of general formula (40) by the sequence described in Scheme 1.

The compounds and processes of the present invention will be better understood by reference to the following examples, which are intended as an illustration of and not a limitation upon the scope of the invention. Further, all citations herein are incorporated by reference.

### EXAMPLE 1

### 2-Dimethylamino-1H-pyrrolo[3,2-b]pyridine-3-carbonitrile

### Example 1A

### Cyano-(3-nitropyridin-2-yl)-acetic acid ethyl ester

To a stirred solution of potassium t-butoxide (11.8 g, 105.2 mmol) in t-butanol (90 mL) and isopropanol (150 mL) was added ethyl cyanoacetate (12.3 g, 108.7 mmol). After 5 min, 2-chloro-3-nitropyridine (10.0 g, 63.1 mmol) was added and the mixture heated to reflux for 6 h. The dark red solution was cooled to room temperature and solvents removed at the pump. The residue was washed with 1 M hydrochloric acid, water and recrystallized twice from methanol to give the title compound (11.0 g, 74%) as crystals. mp 134-135° C. (lit. value 136-137° C.; R. E. Willette, J. Chem. Soc., 1965, 5874). 1H NMR (300 MHz, CDCl₃) tautomers δ 1.35 (2 x t, J 7.1, 3H), 4.32 (2 x q, J 7.1, 2H), 5.88 (s, 0.4H enol), 6.74 (m, 0.6H), 7.66 (dd, J 8.5, 4.7, 0.4H enol), 7.84 (td, J 6.1, 1.4, 0.6H), 8.03 (dd, J 7.5, 1.4, 0.6H), 8.57 (dd, J 8.1, 1.4, 0.4H enol), 8.92 (dd, J 4.7, 1.7, 0.4H enol); MS ESI/APCI+) m/z 236 (M+H)⁺; MS (ESI/APCI−) m/z 234 (M−H)⁻.

### EXAMPLE 1B

### Cyano-(3-aminopyridin-2-yl)-acetic acid ethyl ester

A solution of cyano-(3-nitropyridin-2-yl)-acetic acid ethyl ester (6.00 g, 25.5 mmol) in ethanol (120 mL) was hydrogenated at 60 psi in the presence of palladium on carbon (10%, 0.60 g). After 3 h, the suspension was filtered and solvents removed at the pump to give the title compound (5.00 g, 99%) as a solid. mp 118-119° C. (lit. value 115-117° C.; N. Finch, M. M. Robison, M. P. Valerio, J. Org. Chem., 1972, 37 (1), 51). ¹H NMR (300 MHz, CDCl₃) enol δ 1.35 (t, J 7.1, 3H), 4.27 (q, J 7.1, 2H), 4.88 (s, 2H), 6.64 (m, 1H), 6.78 (dd, J 8.1, 1.4, 1H), 7.21 (td, J 6.1, 1.4, 1H); MS ESI/APCI+) m/z 206 (M+H)⁺; MS (ESI/APCI−) m/z 204 (M−H)⁻.

### EXAMPLE 1C

### 2-Hydroxy-1H-pyrrolo[3,2-b]pyridine-3-carbonitrile

A mixture of cyano-(3-aminopyridin-2-yl)-acetic acid ethyl ester (4.80 g, 23.4 mmol) and xylene (150 mL) was heated to reflux for 16 h. The solution was cooled to 0° C. and filtered to give a dark tan solid. The residue was dissolved in sodium hydroxide solution (3%, 80 mL) and filtered through activated charcoal. Carbon dioxide was bubbled through the solution and filtration gave the title compound (1.71 g, 46%) as a pale solid. mp>250° C. (lit. value>325° C.; N. Finch, M. M. Robison, M. P. Valerio, J. Org. Chem., 1972, 37 (1), 51); ¹H NMR (300 MHz, DMSO-d₆) δ 6.60 (dd, J 7.5, 5.8, 1H), 6.88 (dd, J 7.5, 1.3, 1H), 7.59 (dd, J 5.8, 1.3, 1H); MS (ESI/APCI+) m/z 160 (M+H)⁺; MS (ESI/APCI−) m/z 158 (M−H)⁻.

### A-397031.1 EXAMPLE 1D

### 2-Chloro-1H-pyrrolo[3.2-b]pyridine-3-carbonitrile.

A mixture of 3-cyano-4-azaoxindole (5.00 g, 28.1 mmol) and phosphorus oxychloride (5.00 g, 28.1 mmol) was heated to 105° C. for 2 h. The dark homogenous solution was cooled to room temperature, and excess phosphorus oxychloride removed at the pump. The residue was quenched by the cautious addition of saturated sodium bicarbonate solution (60 mL). The mixture was washed with water, dichloromethane-methanol (4:1) and dried under vacuum to give the title compound (2.50 g, 45%) as a solid. mp>250° C.; ¹H NMR (300 MHz, DMSO-d₆) δ 7.25 (dd, J 7.8, 5.1, 1H), 7.89 (dd, J 7.8, 1.4, 1H), 8.39 (dd, J 5.1, 1.4, 1H); MS (ESI+) m/z 178 (M+H)⁺; MS (ESI−) m/z 176 (M−H)⁻; Anal. Calcd for C₈H₄ClN₃.0.65HCl: C, 47.96; H, 2.29; N, 20.57. Found: C, 47.74; H, 2.33; N, 20.88.

### EXAMPLE 1E

### 2-Dimethylamino-1H-pyrrolo[3,2-b]pyridine-3-carbonitrile

A mixture of 2-chloro-1H-pyrrolo[3,2-b]pyridine-3-carbonitrile (75 mg, 0.42 mmol) and dimethylamine (95 mg, 2.10 mmol, 5 equivalents) was heated to 110° C. for 18 h. The excess amine was removed by vacuum distillation. The residue was purified by reverse-phase chromatography using ammonium acetate as eluant to provide the title compound (32 mg, 40%) as a solid. ¹H NMR (300 MHz, DMSO-d₆) δ 3.22 (s, 6H), 6.93 (dd, J 7.8, 4.7, 1H), 7.41 (dd, J 8.1, 1.4, 1H), 8.10 (dd, J 4.7, 1.4, 1H), 11.20 (s, 1H); MS (ESI+) m/z 187 (M+H)⁺; MS (ESI−) m/z 185 (M−H)⁻; Anal. Calcd for C₁₀H₁₀N₄.0.05 CH₃CO₂H: C, 64.11; H, 5.43; N, 29.61. Found: C, 63.75; H, 5.41; N, 29.52.

### EXAMPLE 2

### 2-Diethylamino-1H-pyrrolo[3,2-b]pyridine-3-carbonitrile

2-Chloro-1H-pyrrolo[3,2-b]pyridine-3-carbonitrile and diethylamine were processed as described in Example 1E to provide the title compound (18.7 mg, 30%) as a solid. ¹H NMR (300 MHz, CD₃OD) δ 1.33 (t, J 7.1, 6H), 3.67 (q, J 7.1, 4H), 6.97 (dd, J 7.8, 5.1), 7.43 (dd, J 6.6, 1.2, 1H), 8.05 (dd, J 5.3, 1.2, 1H); MS (ESI+) m/z 215 (M+H)⁺; MS (ESI−) m/z 213 (M−H)⁻; Anal. Calcd for C₁₂H₁₄N₄: C, 67.27; H, 6.57; N, 26.15. Found: C, 66.89; H, 6.41; N, 25.93.

### EXAMPLE 3

### 2-[(2-Dimethylamino-ethyl)-methyl-amino]-1H-pyrrolo[3,2-b]pyridine-3-carbonitrile

2-Chloro-1H-pyrrolo[3,2-b]pyridine-3-carbonitrile and (2-dimethylaminoethyl) methylamine were processed as described in Example 1E to provide the title compound (83.2 mg, 80%) as a solid. ¹H NMR (300 MHz, CD₃OD) δ 1.96 (s, 3H), 2.49 (s, 6H), 2.87 (t, J 7.2, 7.1, 2H), 3.83 (t, J 7.2, 6.7, 2H), 7.00 (dd, J 7.8, 5.1, 1H), 7.47 (dd, J 7.7, 1.2, 1H), 8.07 (dd, J 5.1, 1.4, 1H); MS (ESI+) m/z 244 (M+H)⁺; MS (ESI−) m/z 242 (M−H)⁻; Anal. Calcd for C₁₃H₁₇N₅. 0.1 CH₃CO₂H: C, 63.59; H, 7.03; N, 28.09. Found: C, 63.64; H, 6.90; N, 28.23.

### EXAMPLE 4

### 2-[(2-Hydroxy-ethyl)-propyl-amino]-1H-pyrrolo[3,2-b]pyridine-3-carbonitrile

2-Chloro-1H-pyrrolo[3,2-b]pyridine-3-carbonitrile and 2-propylamino-ethanol were processed as described in Example 1E to provide the title compound (17.8 mg, 4%) as a solid. ¹H NMR (300 MHz, CD₃OD) δ 1.01 (t, J 7.5, 3H), 1.80 (m, 2H), 3.66 (m, 2H), 3.79 (dt, J 5.2, 35.3, 4H), 6.97 (dd, J 5.1, 7.7, 1H), 7.44 (dd, J 1.4, 7.9, 1H), 8.05 (dd, J 1.3, 5.1, 1H); MS (ESI+) m/z 245 (M+H)⁺; MS (ESI−) m/z 243 (M−H)⁻; Anal. Calcd for C₁₃H₁₆N₄O: C, 63.92; H, 6.60; N, 22.93. Found: C, 63.57; H, 6.58; N, 22.60.

### EXAMPLE 5

### 2-Pyrrolidin-1-yl-1H-pyrrolo[3,2-b]pyridine-3-carbonitrile.

2-Chloro-1H-pyrrolo[3,2-b]pyridine-3-carbonitrile and pyrrolidine were processed as described in Example 1E (with the replacement of ammonium acetate with trifluoroacetic acid as eluant) to provide the title compound (25 mg, 21%) as crystals. ¹H NMR (300 MHz, DMSO-d₆) δ 2.03 (m, 2H), 3.72 (m, 2H), 7.23 (dd, J 8.0, 5.1, 1H), 7.82 (dd, J 8.0, 1.4, 1H), 8.12 (dd, J 5.1, 1.4, 1H), 12.30 (br s, 1H); MS (ESI+) m/z 213 (M+H)⁺; MS (ESI−) m/z 211 (M−H)⁻; Anal. Calcd for C₁₂H₁₂N₄.1.4CF₃CO₂H: C, 47.80; H, 3.63; N, 15.07. Found C, 47.86; H, 3.38; N, 14.68.

### EXAMPLE 6

### 2-(2,5-Dihydro-pyrrol-1-yl)-1H-pyrrolo[3,2-b]pyridine-3-carbonitrile.

2-Chloro-1H-pyrrolo[3,2-b]pyridine-3-carbonitrile and 3-pyrroline were processed as described in Example 1E to provide the title compound (15 mg, 30%) as crystals. ¹H NMR (300 MHz, CD₃OD) δ 4.54 (m, 2H), 3.72 (s, 4H), 6.01 (s, 2H), 7.03 (dd, J 8.0, 5.1, 1H), 7.52 (dd, J 8.0, 1.4, 1H), 8.07 (dd, J 5.1, 1.4, 1H); MS (ESI+) m/z 211 (M+H)⁺; MS (ESI−) m/z 209 (M−H)⁻; Anal. Calcd for C₁₂H₁₀N₄.0.55CH₃OH: C, 66.15; H, 5.40; N, 24.59. Found C, 66.40; H, 5.17; N, 24.47.

### EXAMPLE 7

### (R)-2-(3-Hydroxy-pyrrolidin-1-yl)-1H-pyrrolo[3,2-b]pyridine-3-carbonitrile

2-Chloro-1H-pyrrolo[3,2-b]pyridine-3-carbonitrile and (R)-3-hydroxypyrrolidine were processed as described in Example 1E to provide the title compound (57 mg, 44%) as a solid. [α]²⁰D−20.9 (c 0.75, MeOH); ¹H NMR (300 MHz, CD₃OD) δ 2.00-2.25 (m, 4H), 3.82 (m, 2H), 4.55 (m, 1H), 6.96 (dd, J 8.0, 5.1, 1H), 7.42 (dd, J 8.0, 1.4, 1H), 8.04 (dd, J 5.1, 1.4, 1H); MS (ESI+) m/z 229 (M+H)⁺; MS (ESI−) m/z 227 (M−H)⁻; Anal. Calcd for C₁₂H₁₂N₄O.0.3H₂O: C, 61.69; H, 5.44; N, 23.98. Found: C, 61.39; H, 5.07; N, 23.74.

### EXAMPLE 8

### (S)-2-(3-Hydroxy-pyrrolidin-1-yl)-1H-pyrrolo[3,2-b]pyridine-3-carbonitrile

2-Chloro-1H-pyrrolo[3,2-b]pyridine-3-carbonitrile and (S)-3-hydroxypyrrolidine were processed as described in Example 1E to provide the title compound (48 mg, 37%) as a solid. [α]²⁰_{D}+21.8 (c 0.75, MeOH); ¹H NMR (300 MHz, CD₃OD) δ 2.00-2.25 (m, 4H), 3.82 (m, 2H), 4.55 (m, 1H), 6.96 (dd, J 8.0, 5.1, 1H), 7.42 (dd, J 8.0, 1.4, 1H), 8.04 (dd, J 5.1, 1.4, 1H); MS (ESI+) m/z 229 (M+H)⁺; MS (ESI−) m/z 227 (M−H)⁻;

### EXAMPLE 9

### (R)-2-(2-Hydroxvmethyl-pyrrolidin-1-yl)-1H-pyrrolo[3,2-b]pyridine-3-carbonitrile.

2-Chloro-1H-pyrrolo[3,2-b]pyridine-3-carbonitrile and (R)-2-hydroxymethyl-pyrrolidine were processed as described in Example 1E to provide the title compound (34 mg, 50%) as crystals. mp 207-209° C.; [α]²²_{D}+99.8 (c 0.4, MeOH); ¹H NMR (300 MHz, CD₃OD) δ 2.12 (m, 4H), 3.70 (m, 3H), 3.84 (m, 1H), 4.30 (m, 1H), 6.98 (dd, J 8.0, 5.1, 1H), 7.43 (dd, J 8.0, 1.4, 1H), 8.04 (dd, J 5.1, 1.4, 1H); MS (ESI+) m/z 243 (M+H)⁺; MS (ESI−) m/z 241 (M−H)⁻; Anal. Calcd for C₁₃H₁₄N₄O.0.15CH₃OH: C, 63.92; H, 5.96; N, 22.68. Found: C, 63.81; H, 5.81; N, 22.70.

### EXAMPLE 10

### (S)-2-(2-Hydroxymethyl-pyrrolidin-1-yl)-1H-pyrrolo[3,2-b]pyridine-3-carbonitrile.

2-Chloro-1H-pyrrolo[3,2-b]pyridine-3-carbonitrile and 2-hydroxymethyl-pyrrolidine were processed as described in Example 1E to provide the title compound (87 mg, 64%) as crystals. mp 207-209° C.; [α]²²_{D}+97.6 (c 0.4, MeOH); ¹H NMR (300 MHz, CD₃OD) δ 2.12 (m, 4H), 3.70 (m, 3H), 3.84 (m, 1H), 4.30 (m, 1H), 6.98 (dd, J 8.0, 5.1, 1H), 7.43 (dd, J 8.0, 1.4, 1H), 8.04 (dd, J 5.1, 1.4, 1H); MS (ESI+) m/z 243 (M+H)⁺;MS (ESI−) m/z 241 (M−H)⁻; Anal. Calcd for C₁₃H₁₄N₄O.0.15CH₃OH: C, 63.92; H, 5.96; N, 22.68. Found: C, 63.88; H, 5.63; N, 22.47.

### EXAMPLE 11

### (S)-1-(3-Cyano-1H-pyrrolo[3,2-b]pyridin-2-yl)-pyrrolidine-2-carboxylic Acid Methyl Ester.

2-Chloro-1H-pyrrolo[3,2-b]pyridine-3-carbonitrile and (S)-pyrrolidine-2-carboxylic acid methyl ester were processed as described in Example 1E to provide the title compound (20 mg, 13%) as a solid. ¹H NMR (300 MHz, CD₃OD) δ 2.10 (m, 1H), 2.20 (m, 1H), 2.45 (m, 2H), 3.90 (m, 2H), 4.43 (s, 3H), 4.30 (m, 1H), 5.03 (m, 1H), 7.33 (dd, J 8.0, 5.1, 1H), 7.83 (dd, J 8.0, 1.4, 1H), 8.08 (dd, J 5.1, 1.4, 1H); MS (ESI+) m/z 271 (M+H)⁺;MS (ESI−) m/z 269 (M−H)⁻.

### EXAMPLE 12

### (R)-1-(3-Cyano-1H-pyrrolo[3,2-b]pyridin-2-yl)-pyrrolidine-2-carboxvlic Acid Methyl Ester.

2-Chloro-1H-pyrrolo[3,2-b]pyridine-3-carbonitrile and (R)-pyrrolidine-2-carboxylic acid methyl ester were processed as described in Example 1E to provide the title compound (20 mg, 13%) as a solid. ¹H NMR (300 MHz, CD₃OD) δ 2.10 (m, 1H), 2.20 (m, 1H), 2.45 (m, 2H), 3.90 (m, 2H), 4.43 (s, 3H), 4.30 (m, 1H), 5.03 (m, 1H), 7.33 (dd, J 8.0, 5.1, 1H), 7.83 (dd, J 8.0, 1.4, 1H), 8.08 (dd, J 5.1, 1.4, 1H); MS (ESI+) m/z 271 (M+H)⁺;MS (ESI−) m/z 269 (M−H)⁻.

### EXAMPLE 13

### (R)-[1-(3-Cyano-1H-pyrrolo[3,2-b]pyridin-2-yl)-pyrrolidin-3-yl]-carbamic acid tert-butyl ester

2-Chloro-1H-pyrrolo[3,2-b]pyridine-3-carbonitrile and (R)-pyrrolidin-3-yl-carbamic acid tert-butyl ester were processed as described in Example 1E to provide the title compound (42 mg, 46%) as a solid. ¹H NMR (300 MHz, CD₃OD) δ 1.41 (s, 9H), 2.04 (m, 1H), 2.30 (m, 1H), 3.55 (m, 1H), 3.70-3.92 (m, 3H), 4.28 (m, 1H), 6.95 (dd, J 8.0, 5.1, 1H), 7.40 (dd, J 8.0, 1.4, 1H), 8.01 (dd, J 5.1, 1.4, 1H); MS (ESI+) m/z 328 (M+H)⁺;MS (ESI−) m/z 326 (M−H)⁻.

### EXAMPLE 14

### (S)-[1-(3-Cyano-1H-pyrrolo[3,2-b]pyridin-2-yl)-pyrrolidin-3-yl]-carbamic acid tert-buty ester

2-Chloro-1H-pyrrolo[3,2-b]pyridine-3-carbonitrile and (S)-pyrrolidin-3-yl-carbamic acid tert-butyl ester were processed as described in Example 1E to provide the title compound (42 mg, 42%) as a solid. ¹H NMR (300 MHz, CD₃OD) δ 1.41 (s, 9H), 2.04 (m, 1H), 2.30 (m, 1H), 3.55 (m, 1H), 3.70-3.92 (m, 3H), 4.28 (m, 1H), 6.95 (dd, J 8.0, 5.1, 1H), 7.40 (dd, J 8.0, 1.4, 1H), 8.01 (dd, J 5.1, 1.4, 1H); MS (ESI+) m/z 328 (M+H)⁺;MS (ESI−) m/z 326 (M−H)⁻.

### EXAMPLE 15

### (R,R)-2-(2,5-Dimethyl-pyrrolidin-1-yl)-1H-pyrrolo[3 2-b]pyridine-3-carbonitrile

2-Chloro-1H-pyrrolo[3,2-b]pyridine-3-carbonitrile and (R,R)-2,5-dimethylpyrrolidine were processed as described in Example 1E to provide the title compound (17 mg, 24%) as a solid. 1H NMR (300 MHz, CD₃OD) δ 1.43 (d, 6H), 1.88 (m, 2H), 2.23 (m, 2H), 4.23 (m, 2H), 6.96 (dd, J 8.0, 5.1, 1H), 7.42 (dd, J 8.0, 1.4, 1H), 8.03 (dd, J 5.1, 1.4, 1H); MS (ESI+) m/z 241 (M+H)⁺;MS (ESI−) m/z 239 (M^{-H)}⁻; HRMS 241.1452 (241.1453 calcd for C₁₄H₁₇N₄).

### EXAMPLE 16

(S, S)-2-(2,5-Bis-methoxymethyl-pyrrolidin-1-yl)-1H-pyrrolo[3,2-b]pyridine-3-carbonitrile.

2-Chloro-1H-pyrrolo[3,2-b]pyridine-3-carbonitrile and (S,S)-2,5-bis-methoxymethylpyrrolidine were processed as described in Example 1E to provide the title compound (29 mg, 17%) as a solid. ¹H NMR (300 MHz, CD₃OD) rotomers δ 2.06 (m, 1H), 2.36 (m, 1H), 3.30 (2 x s, 6H), 3.40 (m, 2H), 3.55 (m, 2H), 4.59 (m, 1H), 7.36 (m, 1H), 7.91 (2 x dd, J 8.0, 1.4, 1H), 8.08, 8.42 (2 x dd, J 5.1, 1.4, 1H); MS (ESI+) m/z 301 (M+H)⁺; MS (ESI−) m/z 299 (M−H)⁻; HRMS 301.1662 (301.1665 calcd for C₁₆H₂₁N₄).

### EXAMPLE 17

### 2-Imidazol-1-yl-1H-pyrrolo[3,2-b]pyridine-3-carbonitrile

2-Chloro-1H-pyrrolo[3,2-b]pyridine-3-carbonitrile and imidazole were processed as described in Example 1E to provide the title compound (43.8 mg, 74%) as a solid. ¹H NMR (300 MHz, CD₃OD) δ 7.29 (m, 1H), 7.40 (dd, J 8.1, 5.1, 1H), 7.89 (m, 1H), 8.08 (dd, J 8.1, 1.0, 1H), 8.30 (dd, J 5.1, 1.0, 1H), 8.47 (s, 1H); MS (ESI+) m/z 210 (M+H)⁺; MS (ESI−) m/z 208 (M−H)⁻; Anal. Calcd for C₁₁H₁₇N₅.0.15 CH₃CO₂H: C, 62.20; H, 3.51; N, 32.09. Found: C, 62.05; H, 3.59; N, 32.25.

### EXAMPLE 18

### 2-Piperidin-1-yl-1H-pyrrolo[3,2-b]pyridine-3-carbonitrile

2-Chloro-1H-pyrrolo[3,2-b]pyridine-3-carbonitrile and piperidine were processed as described in Example 1E to provide the title compound (48 mg, 38%) as crystals. mp 241-242° C.; ¹H NMR (300 MHz, CD₃OD) δ 1.78 (m, 6H), 3.72 (m, 4H), 7.01 (dd, J 8.0, 5.1, 1H), 7.46 (dd, J 8.0, 1.4, 1H), 8.08 (dd, J 5.1, 1.4, 1H); MS (ESI+) m/z 227 (M+H)⁺; MS (ESI−) m/z 225 (M−H)⁻; HRMS 227.1301 (227.1297 calcd for C₁₃H₁₅N₄).

### EXAMPLE 19

### 2-(3-Hydroxy-piperidin-1-yl)-1H-pyrrolo[3 2-b]pyridine-3-carbonitrile

2-Chloro-1H-pyrrolo[3,2-b]pyridine-3-carbonitrile and 3-hydroxypiperidine were processed as described in Example 1E to provide the title compound (68 mg, 66%) as a solid. ¹H NMR (300 MHz, CD₃OD) δ 1.66 (m, 2H), 2.01 (m, 2H), 3.34 (m, partially obscured by solvent peak, 1H), 3.47 (m, 1H), 3.85 (m, 2H), 4.04 (m, 1H), 7.00 (dd, J 8.1, 5.1, 1H), 7.46 (dd, J .8, 1.4, 1H), 8.08 (dd, J 5.1, 1.4, 1H); MS (ESI+) m/z 243 (M+H)⁺; MS (ESI−) m/z 241 (M−H)⁻; Anal. Calcd for C₁₃H₁₄N₄O.0.1 CH₃CO₂⁻⁺NH₄: C, 63.42; H, 5.93; N, 22.97. Found: C, 63.64; H, 5.81; N, 23.02.

### EXAMPLE 20

### 2-(4-Hydroxy-piperidin-1-yl)-1H-pyrrolo[3,2-b]pyridine-3-carbonitrile

2-Chloro-1H-pyrrolo[3,2-b]pyridine-3-carbonitrile and 4-hydroxypiperidine were processed as described in Example 1E to provide the title compound (60 mg, 43%) as a solid. ¹H NMR (300 MHz, CD₃OD) δ 1.66 (m, 2H), 2.02 (m, 2H), 3.49 (m, 2H), 3.93 (m, 1H), 4.05 (m, 2H), 7.01 (dd, J 7.8, 5.1, 1H), 7.47 (dd, J 7.8, 1.4, 1H), 8.10 (dd, J 5.1, 1.4, 1H); MS (ESI+) m/z 243 (M+H)⁺; MS (ESI−) m/z 241 (M−H)⁻; Anal. Calcd for C₁₃H₁₄N₄O: C, 64.45; H, 5.82; N, 23.12. Found: C, 64.21; H, 5.89; N, 22.89.

### EXAMPLE 21

### 1-(3-Cyano-1H-pyrrolo[3,2-b]pyridin-2-yl)-piperidine-4-carboxylic acid methyl ester

2-Chloro-1H-pyrrolo[3,2-b]pyridine-3-carbonitrile and piperidine-4-carboxylic acid methyl ester were processed as described in Example 1E to provide the title compound (12.3 mg, 8%) as a solid. ¹H NMR (300 MHz, CD₃OD) δ 1.84 (m, 2H), 2.08 (m, 2H), 2.74 (m, 1H), 3.38 (m, 2H), 4.18 (m, 2H), 7.02 (dd, J 8.1, 5.1, 1H), 7.48 (dd, J 8.1, 1.4, 1H), 8.10 (dd, J 5.1, 1.4, 1H); MS (ESI+) m/z 285 (M+H)⁺; MS (ESI−) m/z 283 (M−H)⁻; Anal. Calcd for C₁₅H₁₆N₄O₂: C, 63.37; H, 5.67. Found: C, 63.05; H, 5.91.

### EXAMPLE 22

### 2-(4-Methyl-piperazin-1-yl)-1H-pyrrolo[3,2-b]pyridine-3-carbonitrile

2-Chloro-1H-pyrrolo[3,2-b]pyridine-3-carbonitrile and 4-methylpiperazine were processed as described in Example 1E to provide the title compound (44.4 mg, 65%) as a solid. ¹H NMR (300 MHz, CD₃OD) δ 1.98 (s, 3H), 2.67 (t, J 5.1, 4H), 3.76 (t, J 5.1, 4H), 7.05 (dd, J 7.8, 5.1, 1H), 7.51 (dd, J 8.1, 1.4, 1H), 8.12 (dd, J 5.1, 1.4, 1H); MS (ESI+) m/z 242 (M+H)⁺; MS (ESI−) m/z 240 (M−H)⁻; Anal. Calcd for C₂₃H₁₅N₅.0.25 CH₃CO₂H: C, 63.26; H, 6.29; N, 27.32. Found: C, 63.09; H, 6.22; N, 27.25.

### EXAMPLE 23

### 2-Morpholin-4-yl-1H-pyrrolo[3,2-b]pyridine-3-carbonitrile

2-Chloro-1H-pyrrolo[3,2-b]pyridine-3-carbonitrile and morpholine were processed as described in Example 1E to provide the title compound (36 mg, 55%) as a solid. ¹H NMR (300 MHz, CD₃OD) δ 3.69 (m, 4H), 3.86 (m, 4H), 7.05 (dd, 7.8, 5.1, 1H), 7.51 (dd, J 7.8, 1.2, 1H), 8.12 (dd, J 5.1, 1.0, 1H); MS (ESI+) m/z 229 (M+H)⁺; MS (ESI−) m/z 227 (M−H)⁻; Anal. Calcd for C₁₂H₁₂N₄O: C, 63.15; H, 5.30; N, 24.55. Found: C, 62.82; H, 5.38; N, 24.54.

### EXAMPLE 24

### 2-Azepan-1-yl-1H-pyrrolo[3,2-b]pyridine-3-carbonitrile

2-Chloro-1H-pyrrolo[3,2-b]pyridine-3-carbonitrile and cyclohexamethyleneamine were processed as described in Example 1E to provide the title compound (17 mg, 24%) as a solid. ¹H NMR (300 MHz, CD₃OD) δ 1.66 (m, 4H), 1.92 (m, 4H), 3.77 (t, J 6.1, 5.8, 4H), 6.97 (dd, J 8.14, 5.1), 7.43 (dd, J 7.8, 1.4, 1H), 8.06 (dd, J 5.1, 0.7, 1H); MS (ESI+) m/z 241 (M+H)⁺; MS (ESI−) m/z 239 (M−H)⁻; Anal. Calcd for C₁₄H₁₆N₄: C, 69.97; H, 6.71; N, 23.31. Found: C, 69.61; H, 6.60; N, 23.02.

### EXAMPLE 25

### 2-(4-Methyl-[1,4]diazepan-1-yl)-1H-pyrrolo[3,2-b]pyridine-3-carbonitrile

2-Chloro-1H-pyrrolo[3,2-b]pyridine-3-carbonitrile and 4-methylhomopiperazine were processed as described in Example 1E to provide the title compound (42 mg, 57%) as a solid. ¹H NMR (300 MHz, CD₃OD) δ 2.13 (m, 2H), 2.48 (s, 3H), 2.78 (m, 2H), 2.94 (m, 2H), 3.82 (t, J 6.1, 2H), 3.91 (m, 2H), 7.00 (dd, J 8.1, 5.3, 1H), 7.46 (dd, J 8.1, 1.4, 1H), 8.08 (dd, J 5.3, 1.4, 1H); MS (ESI+) m/z 256 (M+H)⁺; MS (ESI−) m/z 254 (M−H)⁻; HRMS (FAB/HR) Calcd for C₁₄H₁₇N₅: 256.1562. Found: 256.1562.

### EXAMPLE 26

### 2-Azocan-1-yl-1H-pyrrolo[3,2-b]pyridine-3-carbonitrile

2-Chloro-1H-pyrrolo[3,2-b]pyridine-3-carbonitrile and cycloheptamethyleneamine were processed as described in Example 1E to provide the title compound (66 mg, 44%) as a solid. ¹H NMR (300 MHz, CD₃OD) δ 1.60 (m, 2H), 1.69 (m, 4H), 1.90 (m, 4H), 3.78 (t, J 5.8, 4H), 6.97 (dd, J 7.8, 5.1, 1H), 7.44 (dd, J 7.8, 1.4, 1H), 8.06 (dd, J 5.1, 1.4, 1H); MS (ESI+) m/z 255 (M+H)⁺; MS (ESI−) m/z 253 (M−H)⁻; HRMS (FAB/HR) Calcd for C₁₅H₁₈N₄: 255.1610. Found: 255.1616.

### EXAMPLE 27

### 2-Azonan-1-yl-1H-pyrrolo[3,2-b]pyridine-3-carbonitrile

2-Chloro-1H-pyrrolo[3,2-b]pyridine-3-carbonitrile and azonane were processed as described in Example 1E to provide the title compound (52 mg, 34%) as a solid. ¹H NMR (300 MHz, CD₃OD) δ 1.54 (m, 4H), 1.79 (m, 4H), 1.91 (m, 4H), 3.78 (m, 4H), 6.98 (dd, J 7.8, 5.0, 1H), 7.46 (dd, J 7.8, 1.2, 1H), 8.07 (dd, J 5.1, 1.3, 1H); MS (ESI+) m/z 269 (M+H)⁺; MS (ESI−) m/z 267 (M−H)⁻; Anal. Calcd for C₁₆H₂₀N₄.0.2 CH₃OH: C, 70.82; H, 7.63; N, 20.39. Found: C, 70.94; H, 7.53; N, 20.18.

### EXAMPLE 28

### 2-(Octahydro-isoquinolin-2-yl)-1H-pyrrolo[3,2-b]pyridine-3-carbonitrile

2-Chloro-1H-pyrrolo[3,2-b]pyridine-3-carbonitrile and decahydroisoquinoline were processed as described in Example 1E to provide the title compound (88 mg, 54%) as a solid. ¹H NMR (300 MHz, CD₃OD) δ 1.09 (m, 0.5H), 1.55 (m, 10H), 1.98 (m 2H), 2.84 (m, 0.5H), 3.23 (m, 0.5H), 3.42 (m, 1H), 4.02 (m, 2H), 4.28 (m, 0.5H), 7.00 (m, 1H), 7.44 (m, 1H), 8.07 (m, 1H); MS (ESI+) m/z 282 (M+H)⁺; MS (ESI−) m/z 279 (M−H)⁻; HRMS (FAB/HR) Calcd for C₁₇H₂₀N₄: 281.1766. Found: 281.1768; Anal. Calcd for C₁₇H₂₀N₄: C, 72.83; H, 7.19. Found: C, 72.73; H, 7.29.

### EXAMPLE 29

### 2-(3-Aza-bicyclo[3.2.2]non-3-yl)-1H-pyrrolo[3,2-b]pyridine-3-carbonitrile

2-Chloro-1H-pyrrolo[3,2-b]pyridine-3-carbonitrile and 3-aza-bicyclo[3.2.2] nonane were processed as described in Example 1E to provide the title compound (92 mg, 58%) as a solid. ¹H NMR (300 MHz, CD₃OD) δ 1.81 (d, J 1.70, 8H), 2.21 (s, 2H), 3.88 (d, J 4.1, 4H), 6.98 (dd, J 7.8, 5.1, 1H), 7.43 (dd, J 7.8, 1.4, 1H), 8.07 (dd, J 5.1, 1.4, 1H) ¹³C NMR (100 MHz, DMSO-d₆) δ 24.1, 24.4, 30.0, 56.1, 115.3 115.5, 117.9, 125.7, 142.1, 148.2, 154.8; MS (ESI+) m/z 267 (M+H)⁺; MS (ESI−) m/z 265 (M−H)⁻; HRMS (FAB/HR) Calcd for C₁₆H₁₈N₄: 267.1610. Found: 267.1607;Anal. Calcd for C₁₆H₁₈N₄: C, 72.15; H, 6.81. Found: C, 72.19; H, 7.40.

### EXAMPLE 30

### 2-Phenylamino-1H-pyrrolo[3,2-b]pyridine-3-carbonitrile.

2-Chloro-1H-pyrrolo[3,2-b]pyridine-3-carbonitrile and aniline (125 mg, 1.40 mmol, 5 equivalents) were processed as described in Example 1E (with the replacement of ammonium acetate with trifluoroacetic acid as eluant) to provide the title compound (29 mg, 44%) as crystals. ¹H NMR (300 MHz, DMSO-d₆) δ 7.21 (m, 2H), 7.37 (m, 2H), 7.43 (m, 2H), 7.75 (d, J 7.9, 1H), 8.22 (d, J 5.2, 1H), 10.36 (br s, 1H); MS (ESI+) m/z 235 (M+H)⁺; MS (ESI−) m/z 233 (M−H)⁻; Anal. Calcd for C₁₄H₁₀N₄: C, 71.78; H, 4.30; N, 23.92. Found: C, 71.51; H, 4.17; N, 23.93.

### EXAMPLE 31

### 2-Benzylamino-1H-pyrrolo[3,2-b]pyridine-3-carbonitrile.

2-Chloro-1H-pyrrolo[3,2-b]pyridine-3-carbonitrile and benzylamine were processed as described in Example 1E to provide the title compound (22 mg, 16%) as crystals. ¹H NMR (300 MHz, DMSO-d₆) δ 4.63 (m, 2H), 7.2 (m, 2H), 7.31 (m, 2H), 7.41 (m, 2H), 7.80 (m, 1H), 8.12 (m, 1H), 9.23 (m, 1H), 12.40 (br s, 1H); MS (ESI+) m/z 249 (M+H)⁺; MS (ESI−) m/z 247 (M−H)⁻; Anal. Calcd for C₁₅H₁₂N₄.1.2CF₃CO₂H: C, 54.27; H, 3.45; N, 14.55. Found: C, 54.40; H, 3.57; N, 14.23.

### EXAMPLE 32

### 2-Amino-1H-pyrrolo[3,2-b]pyridine-3-carboxylic acid ethyl ester.

### EXAMPLE 32-A

### 2-Amino-1-hydroxy-1H-pyrrolo[3,2-b]pyridine-3-carboxylic acid ethyl ester.

A solution of crude (recrystallized material used in Example 1B) cyano-(3-nitropyridin-2-yl)-acetic acid ethyl ester (6.00 g, 25.5 mmol) in ethanol (120 mL) was hydrogenated at 60 psi in the presence of palladium on carbon (10%, 0.60 g). After 3 h, the suspension was filtered and solvents removed at the pump to give an oil. The oil was treated with concentrated hydrochloric acid (50 mL) and volatiles removed at pump to give a solid. Purification by flash chromatography (dichloromethane-methanol 9:1 to 1:4) gave the title compound (4.00 g, 62%) as a solid. ¹H NMR (300 MHz, DMSO-d₆) δ 1.36 (t, J 6.9, 3H), 4.38 (q, J 6.9, 2H), 7.28 (m, 1H), 7.90 (m, 1H), 8.05 (m, 3H); MS ESI/APCI+) m/z 222 (M+H)⁺; MS ESI/APCI−) m/z 220 (M−H)⁻; Anal. Calcd for C₁₀H₁₁N₃O₂.0.7HCl: C, 48.68; H, 4.78; N, 17.03. Found: C, 48.76; H, 4.78; N, 16.74.

### EXAMPLE 32-B

### 2-Amino-1H-pyrrolo[3,2-b]pyridine-3-carboxvlic acid ethyl ester.

2-Amino-1-hydroxy-1H-pyrrolo[3,2-b]pyridine-3-carboxylic acid ethyl ester (200 mg, 0.90 mmol) was dissolved in water (10 mL). To this was added potassium hydroxide solution (1 M, 5 mL) followed by the portionwise addition of Raney-Nickel (1.75 g, 50% slurry in water). The heterogenous slurry was stirred at room temperature for 6 h. The mixture was filtered through a plug of Celite which was washed with water, the filtrate collected and dried under vacuum to give the title compound (83 mg, 45%) as a solid. ¹H NMR (300 MHz, DMSO-d₆) δ 1.23 (t, J 6.9, 3H), 4.18 (q, J 6.9, 2H), 6.05 (br s, 2H), 6.53 (dd, J 7.6, 4.8, 1H), 7.02 (dd, J 7.6, 1.5, 1H), 7.75 (dd, J 4.8, 1.5, 1H); MS (ESI+) m/z 206 (M+H)⁺; MS (ESI−) m/z 204 (M−H)⁻; Anal. Calcd for C₁₀H₁₁N₃O₂.1.65CF₃CO₂H: C, 40.61; H, 3.24; N, 10.68. Found: C, 40.61; H, 3.10; N, 10.59.

Biological Data

### Determination of Large-Conductance Ca²⁺-activated K⁺ Channel Opening Activity by Rubidium (⁸⁶Rb) Efflux Assay.

Compounds were evaluated for BK channel opening activity using a Rubidium (⁸⁶Rb) efflux assay. The HEK293 cells stably transfected with BK_{Ca} α subunits were plated in Dulbecco's Modified Eagle Medium supplemented with 10% fetal bovine serum and 100 μM Zeocin at 60,000 cells/well in 96 well plates for 4 hours. The cells were then spiked with 0.25 μCi/well of ⁸⁶Rb and incubated at 37° C. in an atmosphere of 5% CO₂/95% O₂ for 24 hours. The cells were washed thoroughly and assays were preformed where the cells were challenged with test compounds for 30 minutes. The supernatant was harvested and saved. The cells were then lysed with 1N NaOH and the supernatant was harvested and saved. In both sets of supernatants, liquid scintillation (Ecolume) was added and the 96-well plates were counted on the Packard TopCount.

The compounds of the present invention exhibited a 50% maximal response of membrane hyperpolarization in Rb+ Efflux BK-alpha (as compared to P1075) at doses >10,000 nM. In a prefered range, compounds of the present invention exhibited a 50% maximal response of membrane hyperpolarization of in Rb+ Efflux BK-alpha (as compared to P1075) at doses between 5,000 nM and 10,000 nM. In a most preferred range, compounds of the present invention exhibited a 50% maximal response of membrane hyperpolarization of Rb+ Efflux BK-alpha (as compared to P1075) at doses less than or equal to 5,000 nM.

### Electrophysiological Determination of Ionic Current through Large-Conductance Ca²⁺-activated K⁺ Channels

The ability of compounds described in the present invention to open BK channels and increase whole cell outward (K⁺) BK-mediated currents was assessed by measuring ionic currents from HEK293 cells stably transfected with the BK_{Ca} α subunit. To isolate BK current from native (background, non-BK) current, the specific and potent BK channel-blocking toxin iberiotoxin (IBTX) [Galvez, A., et al., J. Biol. Chem, 265: 11083-11090 (1990)] was employed at a supramaximal concentration (50 nM). The relative contribution of BK channels current to total outward current was determined by subtraction of the current remaining in the presence of IBTX (non-BK current) from the current profiles obtained in all other experimental conditions (control, drug, and wash). It was determined that at the tested concentration the compounds profiled did not effect non-BK native currents in the oocytes. Recordings were accomplished using standard two-electrode voltage clamp techniques [Stuhmer, W., et al., Methods in Enzymology, Vol. 207: 319-339 (1992)]. Fire-polished patch electrodes had a resistance of 2 to 5 MΩ. The intracellular pipette solution contained (mM): KCl, 107; MgCl₂, 1.2; CaCl₂, 1; EGTA, 10; HEPES, 5; ATP, 0.1 (pH 7.2 with KOH; total K ˜140 mM). The bath solution contained (mM): KCl, 5; NaCl, 135; CaCl₂, 2.6; MgCl₂, 1.2; HEPES, 5 (pH 7.4 with NaOH). After a tight-seal was formed, the membrane was ruptured and the capacitance transient was integrated on-line to estimate cell capacitance as a measure of cell size. Uncompensated series resistance was typically 3-10 MΩ. The whole cell currents were amplified using an Axopatch-200B amplifier (Axon Instruments, Foster City, Calif.) and low pass filtered at 5 kHz (−3 dB, 4 pole Bessel filter) before digitization by Digidata 1200B at a sampling rate of 10 kHz.

The compounds of the present invention exhibited a maximal response of ionic currents from HEK293 cells stably transfected with the BK_{Ca} α subunit of >0% at doses of 10,000 nM. In a prefered range, compounds of the present invention exhibited a maximal response of ionic currents from HEK293 cells stably transfected with the BK_{Ca} α subunit of greater than 50% but less than or equal to 100% at doses of 10,000 nM. In a most preferred range, compounds of the present invention exhibited a maximal response of ionic currents from HEK293 cells stably transfected with the BK_{Ca} α subunit of greater than 100% at doses of 10,000 nM.

### In vitro Functional Model

Compounds were evaluated for functional BK channel opening activity using tissue strips obtained from Landrace pig bladders. Landrace pig bladders were obtained from female Landrace pigs of 9-30 kg. Landrace pigs were euthanized with an intraperitoneal injection of pentobarbital solution, Somlethal®, J. A. Webster Inc., Sterling Mass. The entire bladder was removed and immediately placed into Krebs Ringer bicarbonate solution (composition, mM: NaCl, 120; NaHCO₃, 20; dextrose, 11; KCl, 4.7; CaCl₂, 2.5; MgSO₄, 1.5; KH₂PO₄, 1.2; K₂EDTA, 0.01, equilibrated with 5% CO₂/95% O₂ pH 7.4 at 37° C.). Propranolol (0.004 mM) was included in all of the assays to block β-adrenoceptors. The trigonal and dome portions were discarded. Strips 3-5 mm wide and 20 mm long were prepared from the remaining tissue cut in a circular fashion. The mucosal layer was removed. One end was fixed to a stationary glass rod and the other to a Grass FT03 transducer at a basal preload of 1.0 gram. Two parallel platinum electrodes were included in the stationary glass rod to provide field stimulation of 0.05 Hz, 0.5 milli-seconds at 20 volts. This low frequency stimulation produced a stable twitch response of 100-500 centigrams. Tissues were allowed to equilibrate for at least 60 minutes and primed with 80 mM KCl. A control concentration response curve (cumulative) was generated for each tissue using the potassium channel opener P1075 as the control agonist. P1075 completely eliminated the stimulated twitch in a dose dependent fashion over a concentration range of 10⁻⁹ to 10⁻⁵ M dissolved in DMSO using 1/2 log increments. After a 60 minute rinsing period, a concentration response curve (cumulative) was generated for the test agonist in the same fashion as that used for the control agonist P 1075. The maximal efficacy of each compound (expressed as % relative to P1075) is reported. The amount of agent necessary to cause 50% of the agent's maximal response (ED₅₀) was calculated using “ALLFIT” (DeLean et al., Am. J. Physiol., 235, E97 (1980)), hereby incorporated by reference. Agonist potencies were also expressed as an index relative to P1075. The index was calculated by dividing the ED₅₀ for P1075 by the ED₅₀ for the test agonist in a given tissue. Each tissue was used for only one test agonist, and the indices obtained from each tissue were averaged to provide an average index of potency.

The compounds of the present invention exhibited a 50% maximal response of Functional BK Channel Opening Activity in Isolated Bladder Strips at doses >10,000 nM. In a prefered range, compounds of the present invention exhibited a 50% Functional Potassium Channel Opening Activity in Isolated Bladder Strips at doses between 5,000 nM and 10,000 nM. In a most preferred range, compounds of the present invention exhibited a 50% maximal response Functional Potassium Channel Opening Activity in Isolated Bladder Strips at doses less than or equal to 5,000 nM.

As demonstrated by the data, the compounds of the present invention stimulate contractions of the bladder by opening potassium channels and therefore have utility in the treatment of diseases prevented by or ameliorated with potassium channel openers.

The present invention provides pharmaceutical compositions which comprise compounds of formula (I-V) prepared and formulated together with one or more non-toxic pharmaceutically acceptable carriers. The pharmaceutical compositions can be formulated for oral administration in solid or liquid form, for parenteral injection or for rectal administration.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous, intraarticular injection and infusion.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the present invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

The active compounds can also be in micro-encapsulated form, if appropriate, with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions which can be used include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin); f) absorption accelerators such as quatemary ammonium compounds; g) wetting agents such as cetyl alcohol and glycerol monostearate;) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the present invention, stabilizers, preservatives, excipients, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

When used in the above or other treatments, a therapeutically effective amount of one of the compounds of the present invention can be employed in pure form or, where such forms exist, in pharmaceutically acceptable salt, ester or prodrug form. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The compounds of the present invention can be used in the form of pharmaceutically acceptable salts derived from inorganic or organic acids. The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio.

Pharmaceutically acceptable salts are well-known in the art. For example, S. M. Berge et al. describe pharmaceutically acceptable salts in detail in (J. Pharmaceutical Sciences, 1977, 66: 1 et seq). The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. The present invention contemplates pharmaceutically acceptable salts formed at the nitrogen attached to R₂R₃N— group of formula (I-V). The present invention also contemplates a pharmaceutically acceptable di-salt formed at the R₂R₃N— group and the ring nitrogen of formula (I-V). Water or oil-soluble or dispersible products are thereby obtained. Examples of acids which can be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts can be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. The present invention contemplates a pharmaceutically acceptable basic salt formed at the carboxyl group when R₁ is CO₂R₄ and R₄ is hydrogen, or the sulfonyl group when R₁ is SO₂R₄ and R₄ is hydrogen.

The term “pharmaceutically acceptable prodrug” or “prodrug,” as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to compounds of formula (I-V), for example, by hydrolysis in blood.

Typical examples of “pharmaceutically acceptable prodrug” or “prodrug” as used herein, can be made as either ester or other groups know to those skilled in the art that may be hydrolyzed under physiological conditions thus delivering a compound of formula (I-V). Examples of prodrug ester groups include pivoyloxymethyl, acetoxymethyl, phthalidyl, indanyl and methoxymethyl, as well as other such groups known in the art. Other examples of prodrug ester groups can be found in the book (“Pro-drugs as Novel Delivery Systems,” by Higuchi and Stella) cited above. The present invention contemplates a pharmaceutically acceptable prodrug ester to include but not be limited to the formation of a prodrug ester group at the site of R₄ of a compound of formula (I-V) wherein R₁ is selected from the group consisting of CO₂R₄ and SO₂R₄.

The present invention contemplates pharmaceutically active metabolites formed by in vivo biotransformation of compounds of formula (I-V). The term pharmaceutically active metabolite, as used herein, refers to a compound formed by the in vivo biotransformation of compounds of formula (I-V). The present invention contemplates compounds of formula (I-V) and metabolites thereof. A thorough discussion of biotransformation is provided in Goodman and Gilman's, The Pharmacological Basis of Therapeutics, seventh edition, hereby incorporated by reference.

The compounds of the invention, including but not limited to those specified in the examples, possess potassium channel opening activity in mammals (especially humans). As potassium channel openers, the compounds of the present invention are useful for the treatment and prevention of diseases such as asthma, epilepsy, Raynaud's syndrome, impotence, migraine, pain, eating disorders, urinary incontinence, functional bowel disorders, neurodegeneration and stroke.

Actual dosage levels of active ingredients in the pharmaceutical compositions of this invention can be varied so as to obtain an amount of the active compound(s) which achieves the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

The total daily dose of the compounds of this invention administered to a human or lower animal may range from about 0.003 to about 10 mg/kg/day. For purposes of oral administration, more preferable doses can be in the range of from about 0.01 to about 5 mg/kg/day. If desired, the effective daily dose can be divided into multiple doses for purposes of administration; consequently, single dose compositions may contain such amounts or submultiples thereof to make up the daily dose.

Potassium channel openers (KCOs) have been shown to act as smooth muscle relaxants, to hyperpolarize bladder cells and consequently relax bladder smooth muscle cells. Because bladder overactivity and urinary incontinence can result from the spontaneous, uncontrolled contractions of the smooth muscle of the bladder, the ability of potassium channel openers to hyperpolarize bladder cells and relax bladder smooth muscle may provide a method to ameliorate or prevent bladder overactivity. Potassium channel openers have been shown to be useful in the treatment of bladder overactivity, pollakiuria, bladder instability, nocturia, bladder hyperreflexia, urinary incontinence, and enuresis as reported by Andersson, et al., Urology 1997, 50 (Suppl 6A), 74-84; Lawson, et al., Pharmacol. Ther. 1996, 70, 39-63; Nurse, et al., Br. J. Urol., 1991, 68, 27-31; Howe, et al., J. Pharmacol. Exp. Ther., 1995, 274, 884-890; Gopalakrishnan, et al., Drug Development Research, 1993, 28, 95-127. Therefore, the compounds of the present invention, including but not limited to those specified in the examples can be used in the treatment of bladder overactivity, pollakiuria, bladder instability, nocturia, bladder hyperreflexia, urinary incontinence, and enuresis.

Calcium-activated potassium (K_{ca}) channels are a diverse group of ion channels that share a dependence on intracellular calcium ions for activity. The activity of K_{ca} channels is regulated by intracellular [Ca²⁺], membrane potential and phosphorylation. On the basis of their single-channel conductances in symmetrical K⁺ solutions, K_{ca} channels are divided into three subclasses: large conductance (BK)>150 pS; intermediate conductance 50-150 pS; small conductance <50 pS. Large-conductance calcium-activated potassium (Maxi-K or BK) channels are present in many excitable cells including neurons, cardiac cells and various types of smooth muscle cells. [Singer, J. et al., Pflugers Archiv. (1987) 408, 98; Baro, I., et al., Pflugers Archiv. (1989) 414 (Suppl. 1), S 168; and Ahmed, F. et al., Br. J. Pharmacol. (1984) 83, 227]. Maxi-K (BK_{Ca}) channels are activated by an increase in intracellular calcium concentration and membrane depolarization. These channels are sensitive to blockade by iberiotoxin and charybdotoxin. The cloning of multiple splice variants of the pore-forming α-subunit (mSlo, hSlo following the nomenclature of the initially cloned Drosophila slowpoke (dSlo) calcium-activated K⁺ channel) and multiple β subunits has recently generated considerable diversity within the BK_{Ca} family. This, together with the widespread distribution of BK_{Ca} channels throughout the CNS and in peripheral tissues offers rich opportunities for discovering novel therapeutic agents as well as significant challenges in the form of tissue and organ specificity. Therapeutic applications for channel openers have focused on stroke, epilepsy, and bladder overactivity although there is evidence for utility in the treatment of asthma, hypertension, gastric hypermotility, and psychoses (Gribkoff, The Pharmacology and Molecular Biology of Large-Conductance Calcium-Activated (BK) Potassium Channels. In Advances in Pharmacology; Eds.; Academic Press: (1997); pp 319-348; Starrett, Curr. Pharm. Des. (1996), 2, 413-428).

It has been shown in the past that neuronal hyperpolarization can produce analgesic effects. In fact, the opening of potassium channels and the resultanting hyperpolarization in the membrane of target neurons is a key mechanism in the effect of opioids. The peripheral antinociceptive effect of morphine results from activation of ATP-sensitive potassium channels, which causes hyperpolarization of peripheral terminals of primary afferents, leading to a decrease in action potential generation has been reported by Rodrigues, Br., et al., J Pharmacol 2000, 129(1), 110-4. Opening of K_{ATP} channels by potassium channel openers plays an important role in the antinociception mediated by alpha-2 adrenoceptors and mu opioid receptors. KCO's can also potentiate the analgesic action of both morphine and dexmedetomidine via an activation of K_{ATP} channels at the spinal cord level as reported by Vergoni, et al., Life Sci. 1992, 50(16), PL135-8; Asano, et al., Anesth. Analg. 2000, 90(5), 1146-51. Potassium channel openers therefore are useful as analgesics in the treatment of various pain states including but not limited to migraine and dyspareunia as reported by by Lawson, et al., Pharmacol. Ther. 1996, 70, 39-63; Gopalakrishnan, et al., Drug Development Research, 1993, 28, 95-127; Gehlert, et al., Prog. Neuro-Psychopharmacol. & Biol. Psychiat., 1994, 18, 1093-1102. Therefore, the compounds of the present invention, including but not limited to those specified in the examples can be useful as analgesics in the treatment of various pain states including but not limited to migraine and dyspareunia.

The irritative symptoms of BPH (urgency, frequency, nocturia and urge incontinence) have been shown to be correlated to bladder instability as reported by Pandita, et al., The J. of Urology 1999, 162, 943; and treated using potassium channel openers as reported by Andersson; et al., Prostate 1997, 30, 202-215. Therefore, compounds of the present invention, including but not limited to those specified in the examples, can be used to hyperpolarize bladder cells and relax bladder smooth muscle providing a method to ameliorate or prevent the symptoms associated with BPH.

The excitability of corpus cavernosum smooth muscle cells is important in the male erectile process. The relaxation of corporal smooth muscle cells allows arterial blood to build up under pressure in the erectile tissue of the penis leading to erection Andersson, et al., Pharmacological Reviews 1993, 45, 253. Potassium channels play a significant role in modulating human corporal smooth muscle tone, and thus, erectile capacity. Potassium channel openers are smooth muscle relaxants and have been shown to relax corpus cavemosal smooth muscle and induce erections as reported by Andersson, et al., Pharmacological Reviews 1993, 45, 253; Lawson, et al., Pharmacol. Ther., 1996, 70, 39-63, Vick, et al., J. Urol. 2000, 163: 202. Therefore, the compounds of the present invention, including but not limited to those specified in the examples, can be used in the treatment of male sexual dysfunctions such as male erectile dysfunction, impotence and premature ejaculation.

Sexual arousal and excitement are linked to the blood flow to the genital area and lubricate the vagina as a result of plasma transudation. Topical application of KCOs like minoxidil and nicorandil have been shown to increase clitoral blood flow as reported by Kim, J. J., Yu, J. W., Lee, J. G., Moon, D. G., “Effects of topical K-ATP channel opener solution on clitoral blood flow”, J. Urol. 2000, 163 (4), 240. KCO's can be effective for the treatment of female sexual dysfunction including clitoral erectile insufficiency, vaginismus and vaginal engorgement as mentioned in Goldstein, I. and Berman, J. R., “Vasculogenic female sexual dysfunction: vaginal engorgement and clitoral erectile insufficiency syndromes”, Int. J. Impotence Res. 1998, 10, S84-S90, as they increase blood flow to female sexual organs. Therefore, the compounds of the present invention, including but not limited to those specified in the examples, can be used in the treatment of female sexual dysfunction as described herein.

Potassium channel openers may have utility as tocolytic agents to inhibit uterine contractions to delay or prevent premature parturition in individuals or to slow or arrest delivery for brief periods to undertake other therapeutic measures as described in Sanborn, et al., Semin. Perinatol., 1995, 19, 31-40; Morrison, et al., Am. J. Obstet. Gynecol., 1993, 169(5), 1277-85. Potassium channel openers also inhibit contractile responses of human uterus and intrauterine vasculature. This combined effect would suggest the potential use of KCO's for dysmenhorrea as mentioned in Kostrzewska, et al., Acta Obstet. Gynecol. Scand., 1996, 75(10), 886-91. Therefore, since the compounds of the present invention, including but not limited to those specified in the examples relax uterine smooth muscle and intrauterine vasculature they can have utility in the treatment of premature labor and dysmenorrhoea as suggested by Lawson, et al., Pharmacol. Ther., 1996, 70, 39-63.

Potassium channel openers have also been shown to relax gastrointestinal smooth tissues and useful in the treatment of functional bowel disorders such as irritable bowel syndrome Lawson, et al., Pharmacol. Ther., 1996, 70, 39-63. Therefore the compounds of the present invention, including but not limites to those specified in the examples are useful in the treatment of functional bowel disorders such as irritable bowel syndrome.

Potassium channel openers relax airway smooth muscle and induce bronchodilation and are useful in the treatment of asthma and airways hyperreactivity as mentioned by Lawson, et al., Pharmacol. Ther., 1996, 70, 39-63; Buchheit, et al., Pulmonary Pharmacology & Therapeutics 1999, 12, 103; Gopalakrishnan, et al., Drug Development Research, 1993, 28, 95-127. Therefore, the compounds of the present invention, including but not limited to those specified in the examples are useful in the treatment of asthma and airways hyperreactivity.

Epilepsy results from the propagation of nonphysiologic electrical impulses. Potassium channel openers hyperpolarize neuronal cells and lead to a decrease in cellular excitability and have demonstrated antiepileptic effects as demonstrated by Lawson, et al., Pharmacol. Ther., 1996, 70, 39-63; Gopalakrishnan, et al., Drug Development Research, 1993, 28, 95-127; Gehlert, et al., Prog. Neuro-Psychopharmacol. & Biol. Psychiat., 1994, 18, 1093-1102. Therefore, the compounds of the present invention, including but not limited to those specified in the examples can be useful in the treatment of epilepsy.

Neuronal cell depolarization can lead to excitotoxicity and neuronal cell death. When this occurs as a result of acute ischemic conditions, the result is often stroke. Long-term neurodegeneration can bring about conditions such as Alzheimer's and Parkinson's diseases. Potassium channel openers can hyperpolarize neuronal cells and lead to a decrease in cellular excitability. Activation of potassium channels has been shown to enhance neuronal survival. Potasium channel openers have been shown to have utility as neuroprotectants in the treatment of neurodegenerative conditions and diseases such as cerebral ischemia, stroke, Alzheimer's disease and Parkinson's disease as mentioned in Lawson, et al., Pharmacol. Ther., 1996, 70, 39-63; Gopalakrishnan, et al., Drug Development Research, 1993, 28, 95-127; Gehlert, et al., Prog. Neuro-Psychopharmacol & Biol. Psychiat., 1994, 18, 1093-1102; Freedman, et al., The Neuroscientist 1996, 2, 145. Therefore, the compounds of the present invention, including but not limited to those specified in the examples will have utility as neuroprotectants in the treatment of neurodegenerative conditions and diseases such as cerebral ischemia, stroke, Alzheimer's disease and Parkinson's disease.

Potassium channel openers also have utility in the treatment of diseases or conditions associated with decreased skeletal muscle blood flow. These conditions are most often associated with diseases associated with decreased skeletal muscle blood flow such as Raynaud's syndrome and intermittent claudication as described in Lawson, et al., Pharmacol. Ther., 1996, 70, 39-63; Gopalakrishnan, et al., Drug Development Research, 1993, 28, 95-127; Dompeling, et al., Vasa. Supplementum, 1992, 3434; and W09932495. Therefore, the compounds of the present invention, including but not limited to those specified in the examples may be used in the treatment of diseases associated with decreased skeletal muscle blood flow such as Raynaud's syndrome and intermittent claudication.

Potassium channel openers have been shown to be useful in the treatment of eating disorders such as obesity Spanswick, et al., Nature, 1997, 390, 521-25; Freedman, et al., The Neuroscientist, 1996, 2, 145. Therefore the compounds of the present invention, including but not limited to those specified in the examples can be useful in the treatment of eating disorders such as obesity.

Potassium channel openers have been shown to promote hair growth as reported by Lawson, et al., Pharmacol. Ther., 1996, 70, 39-63; Gopalakrishnan, et al., Drug Development Research, 1993, 28, 95-127. Therefore, the compounds of the present invention, including but not limited to those specified in the examples can have utility in the treatment of hair loss and baldness also known as alopecia Potassium channel openers possess cardioprotective effects against myocardial injury during ischemia and reperfusion as mentioned in Garlid, et al., Circ. Res., 1997, 81(6), 1072-82, and have demonstrated an ability to be useful in the treatment of heart diseases Lawson, et al., Pharmacol. Ther., 1996, 70, 39-63; Grover, et al., J. Mol. Cell Cardiol,. 2000, 32, 677. Therefore, the compounds of the present invention, including but not limited to those specified in the examples can be useful in the treatment of heart diseases.

Potassium channel openers, by hyperpolarization of smooth muscle membranes, can exert vasodilation of the collateral circulation of the coronary vasculature leading to increase blood flow to ischemic areas and are thus useful for the treatment of useful for the coronary artery disease as described in Lawson, et al., Pharmacol. Ther., 1996, 70, 39-63; Gopalakrishnan, et al., Drug Development Research, 1993, 28, 95-127. Therefore, the compounds of the present invention, including but not limited to those specified in the examples can be useful for the coronary artery disease.

What is claimed is:

1. A compounds of formula (I): 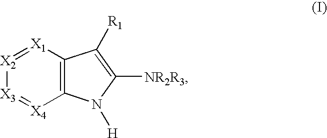
or a pharmaceutically acceptable salt, amide, ester or prodrug thereof, wherein
X₁ is selected from the group consisting of CR_{X1} and N;
X₂ is selected from the group consisting of CR_{X2} and N;
X₃ is selected from the group consisting of CR_{X3} and N;
X₄ is selected from the group consisting of CR_{X4} and N; provided that one or two of X₁, X₂, X₃, X_{X4} is N;
R_{X1}, R_{X2}, R_{X3} and R_{X4} are independently selected from the group consisting of hydrogen, alkenyl, alkenyloxyalkyl, alkoxyalkyl, alkoxycarbonyl, alkoxycarbonylalkyl, alkyl, alkylcarbonyl, alkylcarbonylalkyl, alkylcarbonyloxyalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cyano, cyanoalkyl, cycloalkyl, cycloalkylalkoxyalkyl, cycloalkylalkyl, cycloalkylcarbonyl, cycloalkoxycarbonyl, cycloalkyloxyalkyl, cycloalkylalkylthioalkyl, formyl, halogen, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, hydroxy, hydroxyalkyl, nitro, R_{A}R_{B}N—, (R_{A}R_{B}N)carbonyl, (R_{A}R_{B}N)carbonylalkyl, (R_{A}R_{B}N)sulfonyl, (R_{A}R_{B}N)sulfonylalkyl, alkylsulfonylalkyl, arylalkylSalkyl, arylsulfonylalkyl, heterocyclealkylthioalkyl, HSalkyl, wherein R_{A} and R_{B} are independently selected from the group consisting of hydrogen, alkenyl, alkoxyalkyl, alkoxycarbonyl, alkoxycarbonylalkyl, alkyl, alkylcarbonyl, alkylcarbonylalkyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cycloalkyl, cycloalkylalkoxyalkyl, cycloalkylalkyl, cycloalkylcarbonyl, cycloalkyloxyalkyl, heterocycle, heterocyclealkoxyalkyl, heterocyclealkyl, heterocyclecarbonyl, heterocycleoxyalkyl, hydroxyalkyl;
R₁ is selected from the group consisting of alkylcarbonyl, alkoxycarbonyl, carboxy, carboxyalkyl, cyano, heterocyclecarbonyl, —CO₂R₄, —SO₂R₄, R_{C}R_{D}N—, R_{C}R_{D}Nalkyl, alkylS-, alkylsulfonyl-, (R_{C}R_{D}N)sulfonylalkyl, wherein R_{C} and R_{D} are each independently selected from the group consisting of hydrogen, alkenyl, alkoxyalkyl, alkoxycarbonyl, alkoxycarbonylalkyl, alkyl, alkylcarbonyl, alkylcarbonylalkyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cycloalkyl, cycloalkylalkoxyalkyl, cycloalkylalkyl, cycloalkylcarbonyl, cycloalkyloxyalkyl, heterocycle, heterocyclealkoxyalkyl, heterocyclealkyl, heterocyclecarbonyl, heterocycleoxyalkyl, hydroxyalkyl; and wherein R₄ is selected from the group consisting of hydrogen, alkyl and aryl;
R₂, R₃ are independently selected from the group consisting of hydrogen, alkenyl, alkenyloxyalkyl, alkenyloxy(alkenyloxy)alkyl, alkoxy, alkoxyalkyl, alkoxycarbonyl, alkoxycarbonylalkyl, alkyl, alkylcarbonyl, alkylcarbonylalkyl, alkylcarbonyloxyalkyl, alkylsulfinylalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cyano, cyanoalkyl, cycloalkenyl, cycloalkenylalkyl, cycloalkyl, cycloalkylalkoxyalkyl, cycloalkylalkyl, cycloalkylcarbonyl, cycloalkyloxyalkyl, formyl, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, hydroxy, hydroxyalkyl, mercaptoalkyl, nitro, R_{E}R_{F}N—, (R_{E}R_{F}N)alkyl, (R_{E}R_{F}N)sulfonylalkyl, arylalkylSalkyl, cycloalkylalkylSalkyl, arylsulfonylalkyl, alkylsalkyl alkylsulfonylalkyl, heterocyclealkylthioalkyl; wherein R_{E} and R_{F} are each independently selected from the group consisting of hydrogen, alkenyl, alkoxyalkyl, alkoxycarbonyl, alkoxycarbonylalkyl, alkyl, alkylcarbonyl, alkylcarbonylalkyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cycloalkyl, cycloalkylalkoxyalkyl, cycloalkylalkyl, cycloalkylcarbonyl, cycloalkyloxyalkyl, heterocycle, heterocyclealkoxyalkyl, heterocyclealkyl, heterocyclecarbonyl, heterocycleoxyalkyl, hydroxyalkyl; or
R₂ and R₃ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl, 1,1-dioxidothiomorpholinyl, diazepanyl, azocanyl, azonanyl, isoquinolinyl, or azabicyclononyl, wherein said heterocycle is substituted with 0, 1, or 2 substituents selected from the group consisting of hydrogen, alkenyl, alkenyloxyalkyl, alkoxy, alkoxyalkyl, alkoxycarbonyl, alkoxycarbonylalkyl, alkyl, alkylcarbonyl, alkylcarbonylalkyl, alkylcarbonyloxyalkyl, alkynyl, aryl, arylalkoxyalkyl, arylalkoxycarbonyl, arylalkoxycarbonylalkyl, arylalkyl, arylcarbonyl, arylcarbonylalkyl, arylcarbonyloxyalkyl, aryloxyalkyl, aryloxycarbonyl, aryloxycarbonylalkyl, carboxy, carboxyalkyl, cyano, cyanoalkyl, cycloalkyl, cycloalkylalkoxyalkyl, cycloalkylalkyl, cycloalkylcarbonyl, cycloalkyloxyalkyl, cycloalkylalkylthioalkyl, formyl, halogen, haloalkenyl, haloalkyl, haloalkylcarbonyl, haloalkynyl, heterocycle, heterocyclealkoxyalkyl, heterocyclealkyl, heterocyclecarbonyl, heterocycleoxyalkyl, hydroxy, hydroxyalkyl, nitro, R_{E}R_{F}N—, (R_{E}R_{F}N)carbonyl, (R_{E}R_{F}N)carbonylalkyl, (R_{E}R_{F}N)sulfonyl, (R_{E}R_{F}N)sulfonylalkyl, alkylsulfonylalkyl, arylalkylsalkyl, arylsulfonylalkyl, heterocyclealkylthioalkyl, HSalkyl, wherein R_{E} and R_{F} are defined herein.

2. A compounds of formula (II): 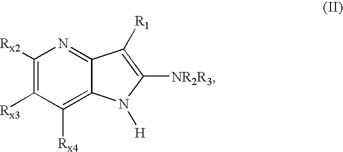
or a pharmaceutically acceptable salt, amide, ester or prodrug thereof, wherein
R_{X2}, R_{X3}, R_{X4}, R₁, R₂, R₃, R₄, R_{A}, R_{B}, R_{C}, R_{D}, R_{E}, R_{F} are as defined in claim 1.

3. A compounds according to claim 2, wherein
R₁ is cyano.

4. A compounds according to claim 2, wherein
R₁ is cyano; and
R₂ and R₃ are each independently selected from the group consisting of hydrogen, alkenyl, alkoxyalkyl, alkoxycarbonyl, alkoxycarbonylalkyl, alkyl, alkylcarbonyl, alkynyl, aryl, arylalkoxycarbonyl, arylalkyl, arylcarbonyl, aryloxycarbonyl, carboxyalkyl, haloalkyl, heterocycle, heterocyclealkyl, heterocyclecarbonyl, hydroxyalkyl, (R_{E}R_{F}N)alkyl.

5. A compounds according to claim 2, wherein
R₁ is cyano; and
R² is alkyl.

6. A compound according to claim 5, selected from the group consisting of
2-Dimethylamino-1H-pyrrolo[3,2-b]pyridine-3-carbonitrile; and
2-Diethylamino-1H-pyrrolo[3,2-b]pyridine-3-carbonitrile.

7. A compounds according to claim 2, wherein
R₁ is cyano; and
R² is hydroxyalkyl.

8. A compound according to claim 7, that is
2-[(2-Hydroxy-ethyl)-propyl-amino]-1H-pyrrolo[3,2-b]pyridine-3-carbonitrile.

9. A compounds according to claim 2, wherein
R₁ is cyano; and
R² is (R_{E}R_{F}N)alkyl.

10. A compound according to claim 9, that is
2-[(2-Dimethylamino-ethyl)-methyl-amino]-1H-pyrrolo[3,2-b]pyridine-3-carbonitrile.

11. A compounds according to claim 2, wherein
R₁ is cyano; and
R² and R³ together with the nitrogen that they are attached to form a heterocycle.

12. A compound according to claim 11, selected from the group consisting of
2-Pyrrolidin-1-yl-1H-pyrrolo[3,2-b]pyridine-3-carbonitrile;
2-(2,5-Dihydro-pyrrol-1-yl)-1H-pyrrolo[3,2-b]pyridine-3-carbonitrile;
(R)-2-(3-Hydroxy-pyrrolidin-1-yl)-1H-pyrrolo[3,2-b]pyridine-3-carbonitrile;
(S)-2-(3-Hydroxy-pyrrolidin-1-yl)-1H-pyrrolo[3,2-b]pyridine-3-carbonitrile;
(R)-2-(2-Hydroxymethyl-pyrrolidin-1-yl)-1H-pyrrolo[3,2-b]pyridine-3-carbonitrile;
(S)-2-(2-Hydroxymethyl-pyrrolidin-1-yl)-1H-pyrrolo[3,2-b]pyridine-3-carbonitrile;
(S)-1-(3-Cyano-1H-pyrrolo[3,2-b]pyridin-2-yl)-pyrrolidine-2-carboxylic acid methyl ester;
(R)-1-(3-Cyano-1H-pyrrolo[3,2-b]pyridin-2-yl)-pyrrolidine-2-carboxylic acid methyl ester;
(R)-[1-(3-Cyano-1H-pyrrolo[3,2-b]pyridin-2-yl)-pyrrolidin-3-yl]-carbamic acid tert-butyl ester;
(S)-[1-(3-Cyano-1H-pyrrolo[3,2-b]pyridin-2-yl)-pyrrolidin-3-yl]-carbamic acid tert-butyl ester;
(R,R)-2-(2,5-Dimethyl-pyrrolidin-1-yl)-1H-pyrrolo[3,2-b]pyridine-3-carbonitrile;
(S, S)-2-(2,5-Bis-methoxymethyl-pyrrolidin-1-yl)-1H-pyrrolo[3,2-b]pyridine-3-carbonitrile;
2-Imidazol-1-yl-1H-pyrrolo[3,2-b]pyridine-3-carbonitrile;
2-Piperidin-1-yl-1H-pyrrolo[3,2-b]pyridine-3-carbonitrile;
2-(3-Hydroxy-piperidin-1-yl)-1H-pyrrolo[3,2-b]pyridine-3-carbonitrile;
2-(4-Hydroxy-piperidin-1-yl)-1H-pyrrolo[3,2-b]pyridine-3-carbonitrile;
1-(3-Cyano-1H-pyrrolo[3,2-b]pyridin-2-yl)-piperidine-4-carboxylic acid methyl ester;
2-(4-Methyl-piperazin-1-yl)-1H-pyrrolo[3,2-b]pyridine-3-carbonitrile;
2-Morpholin-4-yl-1H-pyrrolo[3,2-b]pyridine-3-carbonitrile;
2-Azepan-1-yl-1H-pyrrolo[3,2-b]pyridine-3-carbonitrile;
2-(4-Methyl-[1 ,4]diazepan-1-yl)-1H-pyrrolo[3,2-b]pyridine-3-carbonitrile;
2-Azocan-1-yl-1H-pyrrolo[3,2-b]pyridine-3-carbonitrile;
2-Azonan-1-yl-1H-pyrrolo[3,2-b]pyridine-3-carbonitrile;
2-(Octahydro-isoquinolin-2-yl)-1H-pyrrolo[3,2-b]pyridine-3-carbonitrile; and
2-(3-Aza-bicyclo[3.2.2]non-3-yl)-1H-pyrrolo[3,2-b]pyridine-3-carbonitrile.

13. A compounds according to claim 2, wherein
R₁ is cyano; and
R² is selected from the group consisting of aryl and arylalkyl.

14. A compound according to claim 13, selected from the group consisting of
2-Phenylamino-1H-pyrrolo[3,2-b]pyridine-3-carbonitrile; and
2-Benzylamino-1H-pyrrolo[3,2-b]pyridine-3-carbonitrile.

15. A compounds according to claim 2, wherein
R₁ is cyano; and
R² is hydrogen.

16. A compounds according to claim 2, wherein
R₁ is —CO₂R₄; and
R₂ is alkyl.

17. A compounds according to claim 2, wherein
R₁ is —CO₂R₄; and
R² is hydroxyalkyl.

18. A compounds according to claim 2, wherein
R₁ is —CO₂R₄; and
R² is (R_{E}R_{F}N)alkyl.

19. A compounds according to claim 2, wherein
R₁ is —CO₂R₄; and
R² and R³ together with the nitrogen that they are attached to form a heterocycle.

20. A compounds according to claim 2, wherein
R₁ is CO₂R₄; and
R² is selected from the group consisting of aryl and arylalkyl.

21. A compounds according to claim 2, wherein
R₁ is CO₂R₄; and
R² is hydrogen.

22. A compound according to claim 21, that is
2-Amino-1H-pyrrolo[3,2-b]pyridine-3-carboxylic acid ethyl ester.

23. A compounds of formula (III): 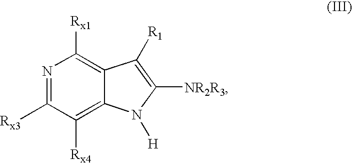
or a pharmaceutically acceptable salt, amide, ester or prodrug thereof, wherein
R_{X1}, R_{X3}, R_{X4}, R₁, R₂, R₃, R₄, R_{A}, R_{B}, R_{C}, R_{D}, R_{E}, R_{F} are as defined in claim 1.

24. A compounds according to claim 23, wherein
R₁ is selected from the group consisting of cyano and —CO₂R₄.

25. A compounds according to claim 23, wherein
R₁ is selected from the group consisting of cyano and —CO₂R₄; and
R² is selected from the group consisting of hydrogen, alkenyl, alkoxyalkyl, alkoxycarbonyl, alkoxycarbonylalkyl, alkyl, alkylcarbonyl, alkynyl, aryl, arylalkoxycarbonyl, arylalkyl, arylcarbonyl, aryloxycarbonyl, carboxyalkyl, haloalkyl, heterocycle, heterocyclealkyl, heterocyclecarbonyl, hydroxyalkyl, (R_{E}R_{F}N)alkyl.

26. A compounds of formula (IV): 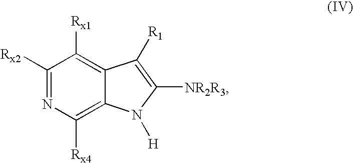
or a pharmaceutically acceptable salt, amide, ester or prodrug thereof, wherein
R_{X1}, R_{X2}, R_{X4}, R₁, R₂, R₃, R₄, R_{A}, R_{B}, R_{C}, R_{D}, R_{E}, R_{F} are as defined in claim 1.

27. A compounds according to claim 26, wherein
R₁ is selected from the group consisting of cyano and —CO₂R₄.

28. A compounds according to claim 26, wherein
R₁ is selected from the group consisting of cyano and —CO₂R₄; and
R² is selected from the group consisting of hydrogen, alkenyl, alkoxyalkyl, alkoxycarbonyl, alkoxycarbonylalkyl, alkyl, alkylcarbonyl, alkynyl, aryl, arylalkoxycarbonyl, arylalkyl, arylcarbonyl, aryloxycarbonyl, carboxyalkyl, haloalkyl, heterocycle, heterocyclealkyl, heterocyclecarbonyl, hydroxyalkyl, (R_{E}R_{F}N)alkyl.

29. A compounds of formula (V): 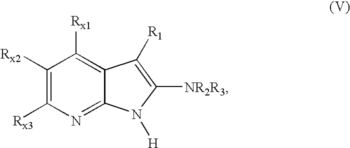
or a pharmaceutically acceptable salt, amide, ester or prodrug thereof, wherein
R_{X1}, R_{X2}, R_{X3}, R₁, R₂, R₃, R₄, R_{A}, R_{B}, R_{C}, R_{D}, R_{E}, R_{F} are as defined in claim 1.

30. A compounds according to claim 29, wherein
R₁ is selected from the group consisting of cyano and —CO₂R₄.

31. A compounds according to claim 29, wherein
R₁ is selected from the group consisting of cyano and —CO₂R₄; and
R² is selected from the group consisting of hydrogen, alkenyl, alkoxyalkyl, alkoxycarbonyl, alkoxycarbonylalkyl, alkyl, alkylcarbonyl, alkynyl, aryl, arylalkoxycarbonyl, arylalkyl, arylcarbonyl, aryloxycarbonyl, carboxyalkyl, haloalkyl, heterocycle, heterocyclealkyl, heterocyclecarbonyl, hydroxyalkyl, (R_{E}R_{F}N)alkyl.

32. A method of claim 1 wherein the disorder is selected from the group consisting of bladder overactivity, pollakiuria, bladder instability, nocturia, bladder hyperreflexia, urinary incontinence, and enuresis.

33. A method of claim 1, wherein the disorder is bladder overactivity.

34. A method of claim 1, wherein the disorder is pain.

35. A method of claim 1, wherein the disorder is BPH.

36. A method of claim 1, wherein the disorder is selected from the group consisting of male erectile dysfunction, impotence and premature ejaculation.

37. A method of claim 1, wherein the disorder is femal sexual dysfunction

38. A method of claim 1, wherein the disorder is selected from the group consisting of premature labor and dysmenorrhoea.

39. A method of claim 1, wherein the disorder is functional bowel disorder.

40. A method of claim 1, wherein the disorder is asthma.

41. A method of claim 1, wherein the disorder is epilepsy.

42. A method of claim 1, wherein the disorder is selected from the group consisting of cerebral ischemia, stroke, Alzheimer's disease and Parkinson's disease.

43. A method of claim 1, wherein the disorder is Raynaud's syndrome.

44. A method of claim 1, wherein the disorder is obesity.

45. A method of claim 1, wherein the disorder is hair loss.

46. A method of claim 1, wherein the disorder is heart diseases.

47. A method of claim 1, wherein the disorder is coronary artery disease.
